(19) United States
(12) Patent Application Publication
(10) Pub. No.: US 2026/0216317 A1
(43) Pub. Date: Jul. 30, 2026

(54) mRNA COMPOSITIONS

(71) Applicant: SISAF LTD, Guildford Surrey (GB)

(72) Inventors: Roghieh Suzanne SAFFIE-SIEBERT, Guildford Surrey (GB); Paulina BARAN-RACHWALSKA, Guildford Surrey (GB); Michael WELSH, Guildford Surrey (GB)

(73) Assignee: SISAF LTD, Guildford Surrey (GB)

(21) Appl. No.: 19/148,564

(22) PCT Filed: Jan. 19, 2024
(86) PCT No.: PCT/GB2024/050155

§ 371 Date: Jul. 16, 2025

(30) Foreign Application Priority Data
Jan. 20, 2023 (GB) 2300915.2

(51) Int. Cl. A61K 39/215; A61K 39/00; A61K 39/02; A61K 39/12; A61K 39/35

(52) U.S. Cl. CPC A61K 39/215; A61K 39/0002; A61K 39/0008; A61K 39/0011; A61K 39/02; A61K 39/12; A61K 39/35; A61K 2039/53; A61K 2039/55555

(57) ABSTRACT

Disclosed is an mRNA vector composition, comprising: mRNA; one or more particles comprising hydrolysable silicon; and one or more lipids, characterised in that the mRNA is mRNA which is not chemically modified. Also disclosed are related products, methods and uses.

### FIELD OF THE INVENTION

The invention relates to mRNA vector compositions and related methods and products. mRNA vector compositions are especially useful in, although not limited to, the field of pharmaceuticals and in particular the field of vaccines.

### BACKGROUND

There is a need for improvements in excipients for biologically active agents. The provision of suitable excipients is required for advances in biomedical research to be fully translated into effective, safe and cost-effective treatments.

mRNA has been proposed as a therapeutic agent. Nucleic acid delivery for therapy or other purposes is well-known, particularly for the treatment of diseases such as cystic fibrosis and certain cancers, and mRNA has been recently used in effective vaccines against SARS-CoV-2.

Delivery systems for delivering nucleic acids to cells fall into three broad classes, namely:
(i) those that involve direct injection of naked nucleic acids,
(ii) those that make use of viruses or genetically modified viruses, and
(iii) those that make use of non-viral delivery agents.

Each has its advantages and disadvantages. Although viruses as delivery agents have the advantages of high efficiency and high cell selectivity, they have the disadvantages of toxicity and triggering of inflammatory responses in particular if treatment requires repeat applications; and are poorly suited to the delivery of large nucleic acids. Accordingly, an mRNA vaccine may advantageously comprise injectable naked mRNA or non-viral delivery systems, such as polyplex vectors or lipid nanoparticle vectors.

Unfortunately, mRNA-based therapies, prophylaxis and vaccinations carry a number of challenges. mRNA is a very fragile molecule. This means that mRNA-based therapies are difficult to store and transport and may for example require storage and transport at refrigeration temperatures, frozen temperatures or ultra-low temperatures. Problems of mRNA transport and storage may be solvable by implementation of cold chain technology. This will, however, increase the cost to the user of the therapy and therefore limit access of the therapy to low and medium income communities, as well as placing strain on the health budgets of wealthier countries.

Lower transfection efficiencies have been noted with non-viral nucleic acid delivery systems. Non-viral nucleic acid delivery systems are based on the compaction of nucleic acid into nanometric particles by electrostatic interaction between the negatively charged phosphate backbone of the nucleic acid and charged polymers, typically cationic lipids and/or peptides (Erbacher, P. et al, Gene Therapy, 1999, 6, 138-145). The mechanism by which these species are introduced into cells is proposed to involve endocytosis of intact complexes, in which complexes formed between the nucleic acid and the lipid become attached to the surface of a cell, then enter the cell by endocytosis. The complex then remains localised within a vesicle or endosome for some time and the nucleic acid component is released into the cytoplasm.

The polymer components of a non-viral delivery system associate electrostatically to form a vector complex. The lipid component shields both the nucleic acid and, to a degree, any peptide component(s) from degradation, endosomal or otherwise; for example, the lipid component may form a lipid bilayer shell which encapsulates other components of the delivery system, including nucleic acid molecules. Cationic lipids for such a use were developed by Felgner in the late 1980s and reported in Proc. Natl. Acad. Sci. USA 84, 7413-7417, 1987; and in U.S. Pat. No. 5,264,618. Felgner developed the now commercially-available cationic liposome known by the trade mark “Lipofectin”. The “Lipofectin” liposome is a spherical vesicle having a lipid bilayer of the cationic lipid DOTMA (2,3-dioleyloxypropyl-1-trimethyl ammonium) and the neutral phospholipid lipid DOPE (phosphatidyl ethanolamine or 1,2-dioleoyl-sn-glycero-3-phosphoethanolamine) in a 1:1 ratio. Various other cationic liposome formulations have since been devised, most of which combine a synthetic cationic lipid and a neutral lipid. In addition to the DOTMA analogues, there may be mentioned complex alkylamine/alkylamides, cholesterol derivatives, such as DC-Chol, and synthetic derivatives of dipalmitol, phosphatidyl-ethanolamine, glutamate, imidazole and phosphonate. However, cationic vector systems vary enormously in their transfection efficiencies in the presence of serum, which clearly impacts on their potential uses for in vivo gene therapy and/or nucleic acid delivery.

A peptide component for use in such complexes typically has two functionalities: a “head group” containing a cell surface receptor-, for example integrin-, recognition sequence, and a “tail” that can bind nucleic acid non-covalently. A peptide component can be designed to be cell-type specific or cell-surface receptor specific. For example a degree of integrin-specificity can confer a degree of cell specificity to the complex. Specificity results from the targeting to the cell-surface receptors (for example, integrin receptors), and transfection efficiencies comparable to some adenoviral vectors can be achieved (Jenkins et al. Gene Therapy 7, 393-400, 2000).

The non-viral delivery of messenger RNA (mRNA) to cells has so far been particularly problematic and limited by the lack of an efficient vector. Attempts to deliver mRNA using known non-viral vehicles that have been used successfully for DNA or siRNA have resulted in sub-optimal levels of protein expression. Furthermore, known non-viral vehicles have poor storage stability when packaged with mRNA.

Therefore, there is a need for vectors that are specifically tailored to the delivery of mRNA, which deliver high levels of mRNA to cells and lead to good levels of protein expression or other RNA activity. There is also a need for compositions tailored to the delivery of mRNA that have good stability upon storage, and mRNA delivery complexes that retain their structure and functionality upon storage at moderate temperatures. There are challenges in maintaing good storage stability, especially at moderate temperatures, such as temperatures of about −5° C. to about 25° C.

A number of mRNA vaccines against SARS-CoV-2, including the Pfizer and BioNTech vaccine BNT162b2 (“Comirnaty”), and the Moderna CX-024414 vaccine, require cold chain storage and transport. This limits accessibility to the vaccine for low-income countries and adds cost and logistical complexity in all markets. It would be advantageous if vaccines could be stored and transported at standard refrigerator temperature (about 5° C.) or room temperature (about 20° C.). It would also be of benefit if vaccines could tolerate higher temperatures (for example, about 30, about 40 or about 50° C.) for storage, or at the very least in the short-term for transport and distribution purposes.

Maintaining mRNA stability in an injectable composition, for example an mRNA vaccine composition, by means of low temperatures, as well as bringing logistical challenges, also has the technical limitation that the mRNA composition must be defrosted before injection, and that after injection it must remain stable in the body for long enough to show sufficient biological activity. This may require stability to be maintained during translocation around the body and/or escape from the endosomal compartment. Stability in vivo must also be maintained for long enough for sufficient translation into protein to take place.

mRNA is vulnerable to degradation, particularly enzymatic degradation. Degradation may be slowed by low temperature and/or by lyophilisation of the nucleic acid (e.g. mRNA), but each of those options come with disadvantages. Short lengths of nucleic acid can be made in an entirely synthetic production environment which can be kept free of degradative enzymes. Such short length nucleic acids may be useful for certain therapeutic applications such as siRNA. Longer nucleic acids cannot be made cost effectively in entirely synthetic production environments and are therefore typically made in production environments which include biologically-sourced material. For example, mRNA may be made by in vitro transcription in which the transcription enzymes derive from biologically sourced material (by biologically sourced material is meant material, particularly nucleic acid, which is either produced in a cell-culture based system, or produced in vitro using synthetic enzymes, for example enzymes produced in a cell-culture based system). mRNA may be extracted from a culture of cells. The use of biologically sourced material increases the chance that biologically-sourced degradative enzymes will be present, unintentionally, in the nucleic acid preparation. This may require extensive purification processes (which are costly and result in loss of yield) and it may not be possible or cost effective to completely remove all degradative enzyme from a biologically-sourced mRNA preparation, for example a preparation of in vitro transcribed (IVT) mRNA or a preparation of DNA such as pDNA. Moreover, during translocation around the body and/or escape from the endosomal compartment, mRNA is exposed to physiological and intracellular conditions, typically including contact with degradative enzymes. Even if a composition successfully removes or inactivates any and all degradative enzymes present, mRNA may remain vulnerable to non-enzymatic degradation.

Encapsulation with lipid or condensation with a peptide or cationic polymer (such as protamine) have been used in the prior art to protect nucleic acid (for example, mRNA for gene therapy or vaccination) from degradation. Condensation with a peptide or other polymer relies on the peptide or polymer remaining intact and retaining its charge. There is a need for improved excipients for increasing the stability of mRNA and for improving its resistance to enzymatic degradation, especially if resistance to enzymatic degradation can be achieved without having to freeze the preparation of nucleic acid. There is also a need for improved excipients for increasing the stability of mRNA, especially when it is to be stored at relatively high temperatures.

Successful formulations for delivering mRNA, for example SARS-CoV-2 mRNA vaccines, typically use modified mRNA. The term “modified” as used in the context of the present disclosure refers to chemical modification to make the RNA more resistant to degradation. For example, chemical modification may include sugar modification, phosphate backbone modification, and base modification.

Unfortunately, as well as potentially increasing cost, chemical modification has the potential to reduce transfection efficiency. When used medically, it may also result in unwanted side-effects such as unwanted immune reactions. By analogy, it is known that chemical modification has the potential to reduce the efficiency of siRNA binding to a sequence of interest to knock down mutant gene expression. When used medically, chemically-modified mRNA may also result in unwanted side-effects such as unwanted immune reactions.

As reported in (https://www.ncbi.nlm.nih.gov/pmc/articles/PMC9326091/), “[t]here is a growing body of evidence suggesting that chemical modifications to mRNA nucleosides impact the speed and accuracy of protein synthesis by the ribosome. Modulations in translation rates have downstream effects beyond protein production, influencing protein folding and mRNA stability”. There is a need for therapeutic mRNA which is non-chemically modified but which also shows sufficient stability when formulated for storage and/or administration. mRNA which is chemically unmodified is used in the compositions of U.S. Pat. Nos. 4,373,071, 4,401,796, 4,415,732, 4,458,066, 4,500,707, 4,668,777, 4,973,679, 5,047,524, 5,132,418, 5,153,319, 5,262,530 and 5,700,642.

### SUMMARY OF THE INVENTION

The present invention is based on an appreciation that particles comprising (or consisting substantially of) hydrolysable silicon, in the presence of one or more lipids, can be used to protect mRNA from degradation over time, even when the mRNA has not been chemically modified, relative to naturally occurring mRNA, to enhance its stability (e.g., chemically modified to protect it from nucleases). Moreover, these advantages can be achieved at relatively high temperatures (during storage or in vivo), as evidenced by Example 6 hereinbelow, wherein chemically unmodified mRNA subjected to a temperature of 37° C. in bovine serum is protected from degradation over time.

In sharp contrast, conventional mRNA delivery systems, such as conventional liposomal mRNA delivery systems, are not sufficiently stabilising; mRNA in them is typically chemically modified, for example modification promoting changes in the mRNA secondary structure, in order to prevent premature degradation of the mRNA (degradation before the mRNA reaches a target site in vivo and is released to have a therapeutic effect).

Thus, the present invention comprehends stabilisation of the mRNA in a pharmaceutical composition (e.g., a formulation for use as or in a vaccine) during storage, prior to the pharmaceutical composition being administered to a subject in need thereof.

The invention also comprehends stabilisation of the mRNA during translocation of the mRNA around the body; and during escape from the endosomal compartment (when mRNA is exposed to physiological conditions, typically including contact with degradative enzymes).

According to a first aspect of the invention, therefore, there is provided an mRNA vector composition comprising:
mRNA;
one or more particles comprising hydrolysable silicon; and
one or more lipids,
characterised in that the mRNA is mRNA which is not chemically modified.

Optionally, the mRNA vector composition further comprises an amino acid, such as glycine. Additionally or alternatively, the composition further comprises one or more disaccharides, such as trehalose. Such components may optionally enhance the stabilising effect of the composition on the mRNA.

According to another aspect of the invention, there is provided a method of increasing the stability of mRNA that is not chemically modified mRNA, comprising the step of bringing it into contact with one or more particles comprising hydrolysable silicon and one or more lipids. It will be appreciated that the one or more particles comprising hydrolysable silicon and one or more lipids may together have one or more features described in relation to the one or more particles comprising hydrolysable silicon and the one or more lipids in the composition according to the first aspect of the invention. Similarly, the mRNA may have one or more features described in relation to the mRNA in the composition according to the first aspect of the invention.

According to a further aspect of the invention, there is provided a pharmaceutical composition comprising the mRNA vector composition according to the first aspect of the invention, wherein the pharmaceutical composition is a prophylactic or therapeutic vaccine composition.

According to another aspect of the invention, there is provided a pharmaceutical composition as described herein, for use as a medicament.

Also provided, in a yet further aspect of the invention, is a method of treating or preventing a disease or disorder, comprising: administering to a subject in need thereof a pharmaceutical composition as described herein.

According to another aspect of the invention, there is provided a method of providing a vaccination to a subject, comprising administration (such as oral (optionally, for stimulation of mucosal or gut associated lymphoid tissue), intranasal, intra-dermal, intra-lymphoid organ administration, direct intra-tumoral administration, ocular, intra-tonsillar, sub-lingual, subcutaneous or intramuscular administration) of a pharmaceutical composition as described herein.

According to a further aspect of the invention, there is provided the use of a pharmaceutical composition as described herein in the manufacture of a medicament, for example a vaccine.

The one or more particles comprising hydrolysable silicon may remove or sequester water molecules, thereby reducing or preventing degradation of the mRNA by hydrolysis.

It has also been found that stabilisation of the mRNA may be due to binding of the mRNA to the silicon particles.

In particular, the one or more particles comprising hydrolysable silicon may electrostatically co-ordinate with the mRNA.

Si-mRNA binding may be especially effective at reducing or preventing mRNA degradation if the silicon particles aggregate into chains of the particles. Thus, preferably, the silicon particles are aggregated.

The chains may extent into the interior of lipidic structures (such as liposomes, incomplete liposomes, micelles, incomplete micelles and/or lipid globules) formed by the one or more lipids. mRNA bound to silicon particles in chains embedded in the interior of lipidic structures may be especially protected from the action of nucleases; and may be especially shielded from water molecules.

The lipidic structures (such as liposomes, incomplete liposomes, micelles, incomplete micelles and/or lipid globules) may typically have a mean diameter in a range of from about 100 nm to about 500 nm.

Meanwhile, the silicon nanoparticles may typically have a mean diameter in a range of from about 1 nm to about 30 nm. This relative difference in size may optimise the formation of aggregates (especially, chains) of the particles being embedded in, and/or decorating the surface of, the lipidic structures.

The formation of silicon particle aggregates embedded in, and/or decorating the surface of, the lipidic structures, may be further promoted by the composition being an extruded composition, especially wherein the particles and lipid(s) have together been extruded before addition of the mRNA (see Example 1 below).

After administration to a patient in need thereof, the silicon may begin, gradually, to be hydrolysed in vivo, thereby eventually releasing intact mRNA at a target treatment site. Thus, transfection efficiency may be enhanced (or at least, not compromised) by the compositions described herein.

A conventional lipid nanoparticle delivery system for mRNA, formulated with lipids (but without hydrolysable silicon) typically has a core encapsulated by a shell. The shell is often a lipid bilayer. The core is typically amorphous and comprises the mRNA, together with water (and sometimes other components, depending on the formulation employed). Although this may protect the mRNA from an external medium, mRNA molecules in the core are nonetheless in contact with water molecules. In such compositions, the core may have a water content of from 10 to 40% by volume.

In sharp contrast to conventional lipid nanoparticles, in the composition according to the first aspect of the present invention the particles comprising hydrolysable silicon shield the mRNA from water molecules (such as by binding electrostatically to the mRNA).

The silicon may also display other mechanisms that sequester water molecules away from the mRNA; e.g., by the silicon itself sacrificially reacting with water, instead of the mRNA doing so.

Thus, preferably, degradation of the mRNA at room temperature (20° C.) is reduced by at least about half, more preferably by a factor of at least about 5, 10, 35, 50, 100, 500 or 1000, compared to an equivalent composition without the silicon particles.

Preferably, the mRNA according to all aspects of the invention has a length of at least about 1000 nucleotides, especially a length in a range of from about 1000 to about 10,000 nucleotides. It may be difficult to make nucleic acids of such lengths cost effectively in entirely synthetic production environments and they may therefore typically be made in production environments which include biologically sourced material, such as by in vitro transcription. They may therefore especially benefit from one or more of the features of all aspects of the invention as described herein.

mRNA which is not Chemically Modified

mRNA according to all aspects of the invention is not chemically modified. The meaning of “not chemically modified” will now be described in detail.

Preferably, at least about 70%, more preferably at least about 80%, of the ribonucleosides of the mRNA have the same molecular structure as naturally occurring ribonucleosides.

Generally, the mRNA according to all aspects of the invention comprises at least one open reading frame. Optionally, each ribonucleoside of the open reading frame has the same or substantially the same molecular structure (the location of atoms relative to each another and the location of bonds) as a ribonucleoside of an open reading frame of a naturally occurring mRNA. (It will be understood that a nucleoside is a nitrogenous base and a carbohydrate group, whereas a nucleotide is a nucleoside with one or more phosphate groups attached.)

Preferably, at least about 70%, more preferably at least about 80%, of the ribonucleosides of the open reading frame of the mRNA have the same molecular structure as naturally occurring ribonucleosides.

Suitably, the ribonucleosides of the mRNA have the same molecular structure as naturally occurring ribonucleosides, except for any uridine present. Modification of uridine may reduce immunogenicity of the mRNA, for example when it is administered as part of a therapeutic composition. However, the ribonucleosides of the mRNA according to all aspects of the invention may have the same molecular structure as naturally occurring ribonucleosides.

Most suitably, each ribonucleoside of the open reading frame has the same molecular structure as a ribonucleoside of an open reading frame of a naturally occurring mRNA, expect for the modification of at least one uridine if uridine is present.

In some embodiments, the mRNA has the same or substantially the same molecular structure as a naturally occurring mRNA, especially a naturally occurring eukaryotic mRNA.

As used herein, the term “naturally occurring” means of natural human or animal origin. It will be understood that a molecular structure that is the same as a naturally occurring molecular structure may nevertheless be synthesized in vitro, such as by in vitro transcription (IVT) as described herein.

The mRNA may suitably comprise a 5′ untranslated region (UTR), an open reading frame (ORF) and a 3′ UTR (the mRNA may comprise more than one 5′ UTR; more than one ORF; and/or more than one 3′UTR). The ribonucleosides of the 5′ UTR, the ORF and the 3′ UTR preferably all have the same molecular structure as naturally occurring ribonucleosides. The mRNA may be linear or circular.

Preferably, the mRNA comprises or consists of a 5′ cap, a 5′ untranslated region (UTR), an open reading frame (ORF), a 3′ UTR and a poly(A) tail.

Preferably, the 5′ cap has the same molecular structure as a naturally occurring 5′ cap, such as Cap 0 or Cap 1. Optionally, the 5′ cap contains phosphodiester analogues (e.g. phosphorothiolate, phosphoroselenoate, boranophosphate, imidodiphosphate); or is an “anti-reverse” cap analogue (ARCA) or a locked nucleic acid (LNA) cap.

Preferably, the poly(A) sitail has the same molecular structure as a naturally occurring poly(A) tail, but it is preferred to have a number in a range of from about 100 to about 150 adenosines in the poly(A) tail.

Optionally, the poly(A) sitail comprises adenosine analogues (e.g. 3′-deoxyadenosine and/or 8-aza-adenosine) and/or a modified phosphate backbone to improve nuclease resistance (e.g. a backbone comprising phosphodiester analogues and/or phosporoamidates).

Optionally, one or both of the 5′ UTR and 3′ UTR contain one or more elements of a globin sequence (suitably, α- or β-globin). Preferably in all aspects, the 5′ UTR contains a Kozak sequence. The Kozak sequence helps guide the ribosome to the correct translation start site.

Linear mRNA is shown in FIG. 19.

Optionally, the mRNA is self-amplifying mRNA, having one or more non-structural gene sequences between the 5′ UTR and the open reading frame. Self-amplifying mRNA is shown in FIG. 20.

Optionally, the mRNA is circular mRNA (circmRNA, or cmRNA), such as that shown in FIG. 21. If the mRNA is cmRNA, it does not have a 5′ cap or poly(A) tail, but it does have a 5′ UTR, ORF and 3′ UTR. In the 5′ UTR is an internal ribosome entry site (IRES) for cap-independent translation.

Preferably, the mRNA has no non-naturally occurring secondary structure, i.e., it has no non-naturally occurring intramolecular base pairing. Such mRNA is more accessible to degradation by nucleases than mRNA having non-naturally occurring secondary structure.

The ability of mRNA vector compositions in accordance with all aspects of the invention to stabilise mRNA that is not chemically modified is illustrated in the Examples, by reference to DasherGFP®, a linear mRNA, which mimics naturally occurring mature mammalian mRNA, having a 5′ Cap 1 structure, a 5′ untranslated region, an open reading frame for green fluorescent protein, a 3′ untranslated region, and a 3′ poly(A) tail (see Examples 4 to 6). Meanwhile, certain disadvantages of highly chemically modified mRNA are evidenced in Example 3.

It will be appreciated that naturally occurring ribonucleosides include A (adenosine), G (guanosine), U (uridine) and C (cytidine). The ribonucleosides occurring naturally in eukaryotes also include m⁵U (5-methyluridine), Gm (2′-O-methylguanosine), f⁶a (N⁶-formyladenosine), m⁵c (5-methylcytidine), ψ (pseudouridine), m³c (3-methylcytidine), m⁶A (N⁶-methyladenosine), m⁶Am (N⁶,2′-O-dimethyladenosine), ac⁴C (N⁴-acetylcytidine), Am (2′-O-methyladenosine), m⁷G (7-methylguanosine), hm⁵C (5-hydroxymethylcytidine), I (inosine) hm⁶A (N⁶-hydroxymethyladenosine), Um (2′-O-methyluridine), hm⁵Cm (2′-O-methyl-5-hydroxymethylcytidine), Cm (2′-O-methylcytidine), m¹A (N¹-methyladenosine).

Thus, the ribonucleosides of the mRNA may be selected from the list consisting of: A (adenosine), G (guanosine), U (uridine), C (cytidine), m⁵U (5-methyluridine), Gm (2′-O-methylguanosine), f⁶a (N⁶-formyladenosine), m⁵c (5-methylcytidine), ψ (pseudouridine), m³c (3-methylcytidine), m⁶A (N⁶-methyladenosine), m⁶Am (N⁶,2′-O-dimethyladenosine), ac⁴C (N⁴-acetylcytidine), Am (2′-O-methyladenosine), m⁷G (7-methylguanosine), hm⁵C (5-hydroxymethylcytidine), I (inosine) hm⁶A (N⁶-hydroxymethyladenosine), Um (2′-O-methyluridine), hm⁵Cm (2′-O-methyl-5-hydroxymethylcytidine), Cm (2′-O-methylcytidine), m¹A (N¹-methyladenosine).

The ribonucleosides of the mRNA may suitably be selected from the list consisting of: A (adenosine), G (guanosine), U (uridine), C (cytidine), m⁶A (N⁶-methyladenosine), m⁶Am (N⁶,2′-O-dimethyladenosine), 8-oxoG (8-oxo-7,8-dihydroguanosine), ψ (pseudouridine), m⁵C (5-methylcytidine), and ac⁴C (N⁴-acetylcytidine), m¹ψ (N¹-methyl-pseudouridine), and mo⁵U (5-methoxyuridine).

The ribonucleosides of the mRNA may most suitably be selected from the list consisting of: A (adenosine), G (guanosine), U (uridine), C (cytidine), ψ (pseudouridine), m¹ψ (N¹-methyl-pseudouridine), and mo⁵U (5-methoxyuridine).

Preferably, none of the ribonucleosides in the mRNA produce non-naturally occurring secondary structure.

The genetic code is degenerate, so that several mRNA codons may correspond to the same amino acid. Codon optimization refers to replacing at least one codon in the ORF with a synonymous codon.

Suitably, the ORF of the mRNA according to all aspects of the invention is or comprises codon-optimized mRNA. Optionally, however, the mRNA according to all aspects of the invention is not or does not comprise codon-optimized mRNA.

Suitably, the mRNA according to all aspects of the invention has the same molecular structure as naturally occurring human or animal mRNA except in that it contains one or more (especially, all) of the following features:
no non-naturally occurring secondary structure;
at least one modified uridine in the ORF;
a codon-optimised ORF;
at least one UTR containing a globin sequence;
either it is circular mRNA or it is linear mRNA having:
a poly(A) tail having a number of adenosines in a range of from about 100 to about 150; and
a 5′ cap that contains phosphodiester analogues, is an “anti-reverse” cap analogue (ARCA) or is a locked nucleic acid (LNA) cap.
   Further Detail on Structural Features of mRNA; on how it May be Made; and on the Function of mRNA

The mRNA may be in vitro transcribed mRNA.

As disclosed herein, the mRNA may comprise a protein-encoding open reading frame and optionally one or more of:
a poly(A) tail;
one or more untranslated regions; and
a 5′ cap structure.

Typically, for a linear mRNA to be functional, it requires modified 5′ and 3′ ends and a coding region (i.e., an open reading frame (ORF) encoding for the protein of interest) flanked by the untranslated regions (UTRs). The nascent mRNA (pre-mRNA), synthetised in the nucleus, undergoes two significant modifications in addition to splicing. During synthesis, a 7-methylguanylate structure, also known as a “cap”, is added to the 5′ end of the pre-mRNA, via 5′→5′ triphosphate linkage. This cap protects the mature mRNA from degradation, and also serves a role in nuclear export and efficient translation. The second modification occurs posttranscriptionally at the 3′ end of the nascent RNA molecule, and is characterized by addition of approximately 200 adenylate nucleotides (poly(A) tail). The addition of the poly(A) tail confers stability to the mRNA, aids in the export of the mRNA to the cytosol, and is involved in the formation of a translation-competent ribonucleoprotein (RNP), together with the 5′ cap structure.

In accordance with normal usage of terms of the art and in accordance with the definitions used in the present disclosure, neither naturally occurring 5′caps nor a 3′poly(A) tail are considered as chemical modification to the synthetised mRNA. The cap and poly(A) tail structures characteristic of mature mRNA can be added during or after IVT synthesis by enzymatic reactions with capping enzymes and poly(A) polymerase, respectively.

In eukaryotic cells, newly synthesized RNA transcripts undergo several nuclear post-transcriptional modifications, known as RNA processing, before they are exported and translated in the cytoplasm. These eukaryotic pre-mRNA modifications include the addition of a cap structure at the 5′-end, the splicing out of introns, the editing of nucleobases and the addition of a poly(A) tail at the 3′-end. RNA capping is a co-transcriptional process that occurs when an RNA molecule is 20-30 nucleotides in length. The cap structure consists of a guanosine residue, harbouring a methylation in the N-7 position, which is bound to the terminal 5′-end nucleotide with a peculiar 5′-5′ triphosphate bridge. This inverted link between the two nucleotides prevents RNA degradation by 5′-3′ exonucleases.

Optionally, the open reading frame of the mRNA encodes an antigen of a pathogen including bacteria, parasites and fungal species as well as viruses. Optionally, the open reading frame of the mRNA encodes a spike protein antigen of a coronavirus such as SARS-CoV-2.

Optionally, in certain preferred embodiments of all aspects of the invention, the open reading frame of the mRNA encodes a tumour-specific antigen. As used herein, the term tumour-specific antigen may refer to an antigen that arises, in one or more malignant cancer cells, from non-synonymous somatic mutation (leading to a neoantigen) or viral-integrated mutation (leading to an oncoviral antigen). Tumor-specific antigens may thus refer to antigens that are completely absent from (not expressed by) non-cancerous (healthy, normal) cells.

Optionally, the open reading frame of the mRNA encodes a tumour-associated antigen. As used herein, the term tumour-associated antigen may refer to an antigen that is over-expressed in a malignant cancer cell, compared to a non-cancerous (healthy, normal) cell, for example due to genetic amplification or post-translational modifications. The term tumour-associated antigen may encompass overexpressed antigens (which term may refer to proteins that are moderately expressed in non-cancerous (healthy, normal) cells, but expressed abundantly in malignant cancer cells); differentiation antigens (which term may refer to proteins that are selectively expressed by the cell lineage from which the malignant cells evolved, an example being prostate-specific antigen); and cancer-germline antigens (which term may refer to antigens that are normally limited to reproductive tissues, but which are aberrantly expressed in a malignant cancer cell; for example, melanoma antigen family A3 (MAGE-A3); New York Esophageal Squamous Cell Carcinoma-1 Antigen (NY-ESO-1); and Preferentially Expressed Antigen in Melanoma (PRAME)).

When the open reading frame of the mRNA encodes a cancer-associated antigen or a cancer-specific antigen, the nucleic acid vector composition may be suitable for use in a prophylactic or therapeutic vaccine composition.

Optionally, the open reading frame of the mRNA encodes an allergen (including but not limited to one or more nut allergens; which in turn include, but are not limited to: one or more seed storage proteins, such as vicilins, legumins, albumins; one or more plant defence related proteins; and one or more profilins).

Optionally, the open reading frame of the mRNA encodes a protein that modulates an immune, autoimmune, or inflammatory disease (including, but not limited to, lupus, atherosclerosis, chronic obstructive pulmonary disease, inflammatory bowel disease, multiple sclerosis, psoriasis, a rheumatic disease, uveitis, atopic dermatitis, and pulmonary fibrosis).

### DESCRIPTION OF THE DRAWINGS

FIG. 1 shows the appearance of silicon wafer before and after milling into powder.

FIG. 2 shows an SEM image of hand-milled silicon particles, which was used to assess their size. Average particle size appears to be 30 nm in this Figure, however particles with sizes of 100 nm have been observed.

FIG. 3 shows a TEM image of the hand milled silicon particles together with lipidic structures, forming SIS0012 of the Examples described hereinbelow.

FIG. 4 shows a TEM image of the silicon particles of FIG. 3 (together with lipidic structures) after complexation with nucleic acid (thus forming nucleic acid-loaded SIS0012 of the Examples described hereinbelow)

FIG. 5 shows an analogous TEM image to that of FIG. 4, except that the particles are particles of more heavily boron-doped silicon.

FIG. 6 shows results for Example 2, namely nucleic acid (mRNA) binding efficiency as determined for silicon-containing delivery vehicle formulations, in accordance with the invention (solid bars); and identical formulations (open bars) except for the absence of the silicon particles.

FIG. 7 shows (from top to bottom): the plate layout and luciferase assay at 6, 24, 48 and 72 h post transfection, for Example 3. In this figure, mRNA1 is labelled “BNT” and mRNA2 is labelled “STech”.

FIGS. 8 and 9 show the assessment, in Example 4, of GFP expression at 24 h post transfection; and relative cell numbers (determined by Hoechst fluorescence; enabling assessment of tolerability/toxicity), following treatment with mRNA complexed with the “Bio-Courier” silicon-containing delivery vehicles in accordance with the invention (in solution: “sshLNP”; and in hydrogel: “ColhySilic”). DasherGFP® mRNA was complexed with the silicon-containing delivery vehicles; or, for comparison, with their liposome-only equivalent, formulated without silicon nanoparticles. Samples were stored at 4° C., room temperature or 37° C. for 0, 24 or 48 h, before transfection of cells. Naked mRNA control (mRNA in nuclease-free water) was subjected to the same storage conditions, and immediately prior to transfection of cells, the naked mRNA control sample was complexed with Lipofectamine 2000 transfection reagent (following the manufacturer's protocol).

FIGS. 10 and 11 show the results of Example 4, a further 24 h after the initial assessment at 24 h shown in FIGS. 8 and 9; thus, FIGS. 10 and 11 respectively show GFP expression at 48h post transfection; and relative cell numbers (determined by Hoechst fluorescence; enabling assessment of tolerability/toxicity).

FIGS. 12 and 13 show raw evaluation of GFP expression (green in original, bright in black and white reproduction) and Hoechst fluorescence (blue in original, not visible in black and white reproduction), at 48 h post transfection following treatment with mRNA complexed with the “Bio-Courier” silicon-containing delivery vehicles in accordance with the invention (in solution: “sshLNP”; and in hydrogel: “ColhySilic”).

FIGS. 14 and 15 show raw evaluation of GFP expression (green in original, bright in black and white reproduction) and Hoechst fluorescence (blue in original, not visible in black and white reproduction), at 48 h post transfection following treatment with mRNA complexed with the liposome-only equivalent of the “Bio-Courier” delivery vehicles, i.e., without silicon present.

FIG. 16 shows results from Example 5, namely a gel retardation assay of naked mRNA (DasherGFP® mRNA) incubated with 2% bovine serum over time. Naked mRNA in nuclease free water was used as control (lane 9) and DNA ladder was used as size guide.

FIG. 17 shows results from Example 5, namely a gel retardation assay of (A) mRNA-SIS0012 incubated with bovine serum (BS) for different durations of time and (B) mRNA extracted from mRNA-SIS0012 before and after incubation with bovine serum. Naked mRNA in nuclease free water was used as a control. DNA ladder was used as a size guide.

FIG. 18 shows a gel retardation assay demonstrating stability of formulated mRNA by nuclease degradation protection assay. The mRNA samples (either naked mRNA or loaded to silicon-containing delivery vehicles) were incubated with 2 or 10% FBS for 1 hr at 37° C. (or left untreated, lane 2 and 5 respectively) and analysed by electrophoresis on a 1% agarose gel. Upper panel shows intact mRNA detected in the loading wells when mRNA was complexed with ProSilic-DSC613G and lower panel shows intact free mRNA (untreated control) and degraded mRNA fragments obtained when samples were exposed to serum nucleases (nucleic acids present in serum showed unspecific smearing band).

FIG. 19 shows a typical structure for naturally occurring linear mRNA.

FIG. 20 shows a typical structure for naturally occurring linear self-amplifying mRNA.

FIG. 21 shows a typical structure for circular mRNA (cmRNA).

### DETAILED DESCRIPTION

Unmodified mRNA (Linear and Circular RNA Molecules)

According to all aspects of the present invention, the mRNA is not chemically modified.

Chemically unmodified mRNA (or more fully, non-chemically modified mRNA) may be linear or circular. Preferably, it is linear.

Optionally, the mRNA is biologically sourced mRNA. As used herein, “biologically sourced” may mean the mRNA is produced in a cell-culture based system or the mRNA is produced in vitro using enzymes produced in a cell-culture based system. Biologically sourced mRNA may include mRNA extracted from cellular material or transcribed in vitro. Preferably, the mRNA is in vitro transcribed (IVT) mRNA, as that term is commonly used in the art.

As used herein, “non-chemically modified mRNA” means that the mRNA does not include chemical changes to the molecular structure of the mRNA. As used herein, the term “chemical changes” may refer to changes which do not occur in nature but which are the deliberate result of a manufacturing process, such as in a laboratory or factory.

Optionally, the isolated mRNA (i.e., when not part of a composition according to the various aspects of the invention) has a half-life upon storage in aqueous solution at about 25° C. of at most one week, measurable by NMR or by GC-MS; and/or has a half-life in vivo of under about an hour, especially under about 10 minutes, which may suitably be measurable by assay of a biological sample. It will be appreciated that the term “half-life in vivo”, as used herein, may refer to the elimination half-life in vivo of the mRNA, i.e. the time period taken for the amount of the functional mRNA, once administered, to reduce by about half. It will also be appreciated that the term “amount of the mRNA” may refer to the amount of the mRNA or a derivative thereof having the same or substantially the same intended pharmaceutical effect. It will be appreciated that the term “functional mRNA” refers to mRNA that is translatable by ribosomes.

It will also be understood that mRNA in which merely the order of nucleotides (A, U/T, G, C), i.e. the sequence of bases which are present, has been modified compared to a naturally occurring sequence of nucleotides, are included within the scope of the invention. Such encompassed changes include codon optimisation and introduction of altered or alternative or mutated promotors or other non-transcribed regions, and also the addition of poly-A tails.

mRNA having a 5′cap may suitably be encompassed by all aspects of the invention. A 5′ cap is a natural feature of mature non-chemically modified naturally occurring functional mRNA. The presence of both a 5′ cap (at the mRNA 5′ end) and a 3′ poly(A) tail (at the 3′ end) may promote efficient translation in eukaryotic cells. The 5′ cap may help to recruit relevant cellular proteins and may mediate cap-related biological functions such as pre-mRNA processing, nuclear export and cap-dependent protein synthesis. All this is largely attributed to protein factors that bind specifically to the cap structure: the cap-binding complex (CBC) in the nucleus and eIF4E in the cytoplasm. For example, a 7-methylguanylate cap (m7G) may play a major role in the coordination of various functional processes that take place throughout the life cycle of mRNA.

The 5′ cap may be recognised by cells as a marker of an actively translating mRNA. It may also play a role as an identifier of self RNA by the innate immune system against foreign RNA. See Nucleic Acids Research, 2016, Vol. 44, No. 16 7511-7526 doi: 10.1093/nar/gkw551 mRNA modifications taking the nucleic acid outside of the scope of the present invention may, in some embodiments, include methylated purines or pyrimidines (except when they solely exist as part of the 5′cap), acylated purines or pyrimidines, or other heterocycles (except when they solely exist as part of the 5′cap).

Modified nucleosides or nucleotides taking the nucleic acid outside of the scope of the present invention may include modifications of the sugar moiety, e.g., wherein one or more of the hydroxyl groups are replaced with a halogen, an aliphatic group, or are functionalized as ethers, amines, or the like.

Thus, the chemical modification of one or more sugar moieties (e.g., wherein one or more of the hydroxyl groups are replaced with a halogen, an aliphatic group, or are functionalized as ethers, amines, or the like) is preferably excluded from the present chemically unmodified mRNA.

Chemical modifications taking mRNA outside of the scope of the claims of the invention may include the presence of one or more universal bases. As used herein, the term “universal base” refers to a nucleotide analogue that can hybridize to more than one nucleotide selected from A, U/T, C, and G. The (presently excluded) universal base may be selected from the group consisting of deoxyinosine, 3-ntiropyrrole, 4-nitroindole, 6-nitroindole, 5-nitroindole.

Thus, preferably, the present chemically unmodified mRNA excludes universal bases.

As described herein, modified nucleosides have been used in prior art mRNA (such as that delivered to a patient in need thereof using a conventional liposome composition) to increase nucleic acid stability. Thus, in especially preferred embodiments, the mRNA is not chemically modified in that it does not contain any nucleotides that differ from A, U/T, C, and G.

By way of a non-limiting but representative example, a sequence AAUCG being changed to AUACG is included within the scope of the invention. However, a modification to the G nucleobase of the G nucleotide in the sequence, may be not within the scope of the invention. 5′ cap

As described above, mRNA falling inside the scope of the invention includes mRNA which has a 5′ cap.

The 5′ cap may optionally have the Cap 0 (m⁷GpppN) structure, which requires a 7-methylguanosine connected by a triphosphate bridge to the first nucleotide.

The 5′ cap may optionally have the Cap 1 (m⁷GpppNm) structure, which requires methylation of the 2′-hydroxyl group of the first cap-proximal nucleotide.

The 5′ cap may optionally have the Cap 2 (m⁷GpppNm pN^{m}) structure, having an additional 2′-O-methylation of the second nucleotide.

The 5′ cap may have a modified Cap 0, modified Cap 1, or modified Cap 2 structure. In certain preferred embodiments, for example when the open reading frame of the mRNA encodes a spike protein antigen of SARS-CoV-2, the 5′ cap is Cap 1, or the 5′ cap is a modified Cap 1 having the m⁷G⁺m^{3′}-5′-ppp-5′-Am cap structure.

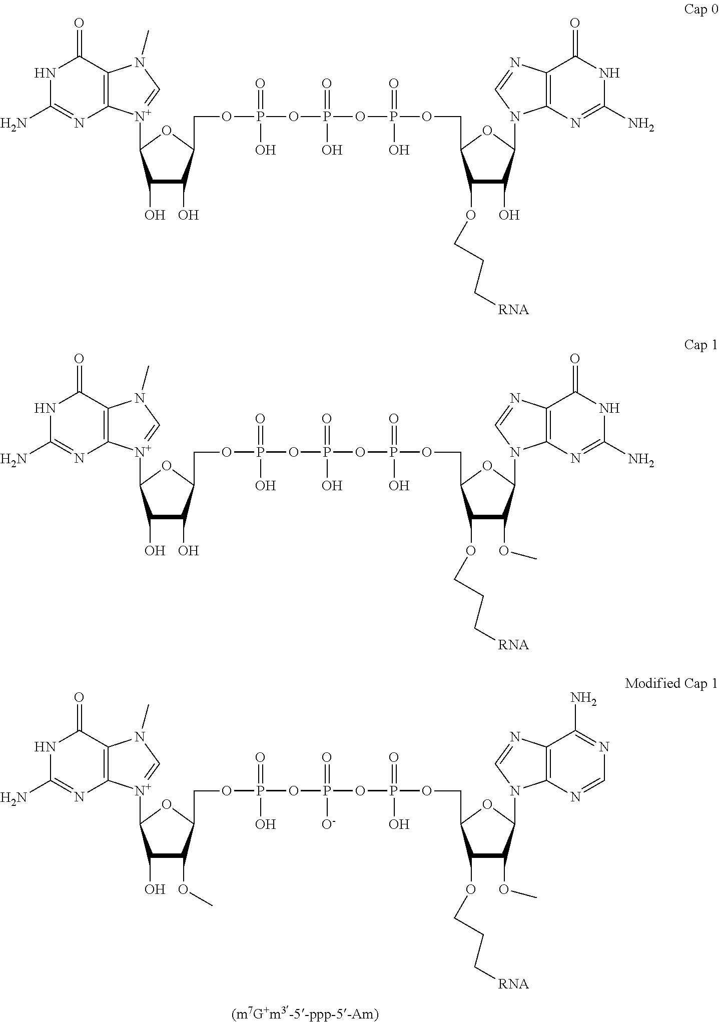

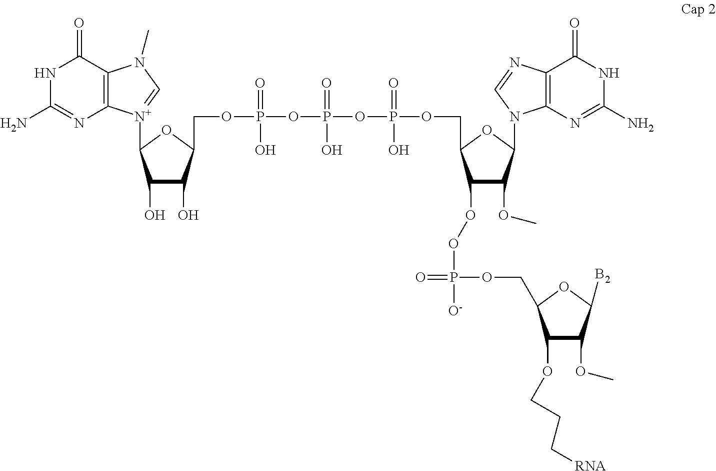

5′ caps may allow a ribosome to recognize the beginning (5′ end) of a molecule of mRNA. 5′ caps may improve translation efficiency by binding to the eukaryotic translation initiation factor 4E (eIF4E).

mRNA, Especially IVT mRNA; and Purification

The mRNA according to all aspects of the invention may be or comprise in vitro transcribed (IVT) mRNA. The IVT mRNA may be as described in WO 2023/002223 A1 (filed by SiSaf Ltd), incorporated herein by reference in its entirety.

Preparing a (linear or circular) mRNA vector composition of the invention may involve purifying the mRNA. In certain embodiments this may require purifying the mRNA from a transcription reaction mixture (which may contain impurities, such as one or more of: enzymes, residual nucleoside triphosphates, DNA template, and aberrant mRNAs formed during IVT) to form a preparation of in vitro transcribed mRNA.

Purification may include, but is not limited to, one or more of: size exclusion chromatography (SEC); ion pair reverse-phase chromatography (IPC); ion exchange chromatography (IEC); affinity based separation; tangential flow filtration (TFF); core bead chromatography; hydroxyapatite chromatography; mRNA precipitation combined with TFF (during TFF, the membrane captures the precipitated mRNA product while other impurities are removed by diafiltration); DNA template removal by performing a digestion with immobilised DNase; and the use of tagged DNA template that can be removed after IVT using affinity chromatography. In certain preferred embodiments, for example when the open reading frame of the mRNA encodes a spike protein antigen of SARS-CoV-2, purification can involve oligo-dT affinity purification, buffer exchange by tangential flow filtration into sodium acetate (pH 5.0) and sterile filtration.

It will be understood that even when purification is complete, a preparation of in vitro transcribed mRNA may still comprise, in at least trace amounts, one or more of: linear DNA; one or more RNA polymerases; and one or more nucleoside triphosphates. For example, a preparation of in vitro transcribed mRNA may comprise at least 0.01% v/v linear DNA. A preparation of in vitro transcribed mRNA may comprise at least 0.01% v/v RNA polymerases. A preparation of in vitro transcribed mRNA may comprise at least 0.01% v/v nucleoside triphosphates.

Especially, the mRNA vector composition may comprise at least 0.01% v/v nucleases. These may be present as residue from biologically sourced materials used to prepare the mRNA. Although these may be present, the particles comprising hydrolysable silicon may protect the mRNA from them. Thus, the advantages described herein may especially apply when the mRNA is in vitro transcribed mRNA.

Preparing the mRNA Vector Composition

In the method of preparing a mRNA vector composition according to the invention, the preparation of mRNA is then combined with water, a particle comprising hydrolysable silicon, and one or more lipids.

The composition may optionally further comprise one of more amino acid (for example glycine or a mixture of nucleic acids including glycine) and optionally one or more non-reducing disaccharides such as trehalose.

A suitable preparation method may include:
dispersing the lipid component in a solvent such as methanol;
generating a thin film of lipid by evaporating the solvent (for example in rotary evaporator); and
hydrating the lipid with an aqueous solution containing the hydrolysable silicon particles (and, optionally, a non-reducing disaccharide such as trehalose and/or one or more amino acid such as glycine).

The composition may optionally be passed though filters, for example 0.4 and 0.1 μm filters, to achieve complexation and dispersal of the particles.

The composition may optionally be stored at 4° C. if required to allow further complexation to take place.

A carrier (i.e., all non-mRNA components of the vector composition defined herein) prepared in this way may then be complexed with mRNA, wherein the mRNA may suitable be provided in nuclease-free water.

The carrier (i.e., all non-mRNA components of the vector composition defined herein) and the mRNA may be combined at ratios by weight of the mRNA to the carrier of about 1:2 1:6 to 1:16. Preferred ratios by weight of the mRNA to the carrier are those in a range of from about 1:8-1:12, where 1:8 usually allows a small excess of the mRNA, and 1:12 usually allows a small excess of the carrier.

mRNA Structure and Coding Functionality

As described, in some embodiments a molecule of mRNA can optionally comprise, in addition to a protein-encoding open reading frame, one or more, or all of: a poly(A) tail; a 5′cap and one or more untranslated regions.

In some embodiments, the protein-encoding open reading frame of the mRNA encodes an antigen, thereby providing a formulation which is a vaccine. The antigen may be a viral antigen, or bacterial, fungal and parasite and tumour associated antigens/neo-antigens, for example an antigen of SARS-CoV-2, for example an antigen which is or which derives from the spike protein of SARS-CoV-2 or a part thereof. Thus, the open reading frame of the mRNA may in some embodiments encode a spike protein antigen of SARS-CoV-2. In some embodiments, the open reading frame can encode a mutated version of a naturally occurring protein. For example, in some embodiments when the open reading frame encodes the spike protein of SARS-CoV-2 or a part thereof, two mutations can be included in which the original amino acids are replaced with prolines. Without wishing to be bound by theory, it is thought that this can ensure the resultant S glycoprotein remains in an antigenically optimal pre-fusion conformation.

In some embodiments, the protein-encoding open reading frame of the mRNA may encode multiple proteins. For example, the protein-encoding open reading frame of the mRNA may encode a viral antigen and an adjuvanting protein, or multiple viral antigens. In some embodiments the mRNA sequence may also include molecular adjuvants to specifically trigger toll-like receptors or other immune activation pathways. In certain preferred embodiments, for example when the open reading frame of the mRNA encodes a spike protein antigen of SARS-CoV-2, the open reading frame may additionally encode a signal peptide, for example a S glycoprotein signal peptide.

### 3′ Poly(A) Tail

Without wishing to be bound by theory, it is also thought that providing the mRNA with a 3′ poly(A) tail can improve translational activities. A poly(A) tail can, in preferred embodiments (for example when the open reading frame of the mRNA encodes a spike protein antigen of SARS-CoV-2) be added to the mRNA during in vitro transcription of the mRNA by including a poly(A) sequence in the DNA template. The tail size can be selected to optimise expression of the mRNA. Advantageously, in vitro transcription of mRNA from a DNA template can produce mRNA having a defined poly(A) tail length. In some embodiments, the poly(A) sitail can have a length of between 100 and 200 nucleotides, for example between 120 and 150 nucleotides. In certain preferred embodiments, for example when the open reading frame of the mRNA encodes a spike protein antigen of SARS-CoV-2, a 110-nucleotide poly(A)-tail is provided having 30 adenosine residues separated by a 10-nucleotide linker sequence from a further 70 adenosine residues. It may be understood that mRNA having a poly(A) tail is not chemically modified.

### Untranslated Regions (UTRs)

Untranslated regions are non-coding regions of the mRNA sequence, located at the upstream (5′ UTR) and downstream (3′ UTR) domains of the mRNA coding region. Without wishing to be bound by theory, it is thought that UTRs can assist with transcription regulation, and that UTRs impact translation efficiency through involvement in translation machinery recognition, recruitment, and mRNA trafficking. It is thought that UTRs can alter mRNA decay and translation efficiency through reactions with RNA binding proteins.

In certain preferred embodiments, for example when the open reading frame of the mRNA encodes a spike protein antigen of SARS-CoV-2, a 5′ UTR is provided which is derived from human alpha-globin mRNA with an optimized Kozak sequence, and/or a 3′ UTR is provided comprising two sequence elements derived from the amino-terminal enhancer of split (AES) mRNA and the mitochondrial encoded 12S ribosomal RNA. Without wishing to be bound by theory, it is thought that such UTRs confer high total protein expression. It may be understood that mRNA having one or more UTRs is not chemically modified.

### Open Reading Frame (ORF)

As the term is used herein, the open reading frame (ORF) refers to the, or one of the, protein-encoding region of the mRNA. In some embodiments, the ORF sequence may include synonymous common codons (and/or codons having higher tRNA abundance) as replacements for rarer codons. It is thought that in this way, highly expressed genes can be translated using the same codons of the host, and/or guarantee the abundance of the relevant tRNA during the expression of the mRNA. However, having a higher translation rate of the nucleic acid may not always be preferred, as some proteins require a low translation rate for proper folding. In these circumstances, using codons with low frequency in ORF may yield higher quality protein products. It will be appreciated that both such approaches are in the scope of the various aspects of the present invention, wherein the mRNA is not chemically modified.

### The One or More Particles Comprising Hydrolysable Silicon

According to all aspects of the invention, the one or more particles, which comprise hydrolysable silicon, may be pure silicon, or another hydrolysable silicon-containing material.

If they are not pure silicon, they contain at least 50% by weight silicon, i.e. they comprise at least 50% by weight silicon atoms based on the total mass of atoms in the particles. For example, the silicon particles may contain at least 60, 70, 80, 90 or 95% silicon.

The silicon particles preferably show a rate of hydrolysis, for example in PBS buffer at room temperature, of at least 10% of the rate of hydrolysis of pure silicon particles of the same dimensions. Assays for hydrolysis of silicon-containing material are widely known in the art (see, for example, WO2011/001456, incorporated by reference herein).

Although particle of the invention may contain some silica, silica is not hydrolysable silicon and at least half of the silicon atoms in the particles are in the form of elemental silicon (or doped elemental silicon). According to all aspects of the invention, the particles comprising hydrolysable silicon may be made of either pure silicon or a hydrolysable silicon-containing material. They are preferably porous.

The particles may, especially, be nanoparticles.

Preferably, the nanoparticles' mean diameter is below 500 nm, such as being below 100, 80, 70, 50 or 30 nm. The mean diameter may be between 20 nm and 40 nm (e.g., about 30 nm).

The nanoparticles may thus have a mean diameter in a range of about 1 nm to about 500 nm, especially about 1 nm to about 250 nm, more especially about 1 nm to about 100 nm, preferably about 1 nm to about 50 nm, especially about 5 nm to about 50 nm, more especially about 1 nm to about 30 nm (such as about 10 nm). Mean diameter may be measured, for example, by scanning electron microscopy (SEM) or by transmission electron microscopy (TEM).

A particularly preferred range for the nanoparticles' mean diameter is about 1 nm to about 30 nm. This may have advantages in terms of the nanoparticles' aggregation, as described herein.

Particles comprising hydrolysable silicon can be made porous by standard techniques such as contacting the particles with a hydrofluoric acid (HF)/ethanol mixture and applying a current. By varying the HF concentration and the current density and time of exposure, the density of pores and their size can be controlled and can be monitored by scanning electron micrography and/or nitrogen adsorption desorption volumetric isothermic measurement.

If the particles are porous, their total surface area will be increased by virtue of their porosity. For example the surface area may be increased by at least 50% or at least 100% over the surface area of a corresponding non-porous particle. In many circumstances porous particles in accordance with all aspects of the invention will in reality have a much greater increase in total surface area by virtue of their porosity. According to certain embodiments the porosity is at least 30, 40, 50 or 60%. This means that, respectively, 30, 40, 50 or 60% of the particle volume in pore space.

Average pore diameter may be in a range of from about 0.1 nm to about 10 nm, for example from about 0.1 nm to about 3 nm, such as about 2 nm.

### Optional Doping of Silicon

All aspects of the present invention optionally concern doped silicon containing material. The silicon may optionally be n-doped or p-doped. The invention includes embodiments in all aspects wherein the silicon is doped with one or more elements selected from Mg, P, Cu, Ga, Al, In, Bi, Ge, Li, Xe, N, Au, Pt. Most preferably the dopant is a p-dopant. Most preferably the dopant is boron. P-doped silicon is especially suitable for stabilising negatively charged nucleic acid. N-doped silicon may also be useful in indirectly stabilising negatively charged nucleic acids because they can protect lipids from degradation which may indirectly increase the stabilization and protection of the mRNA.

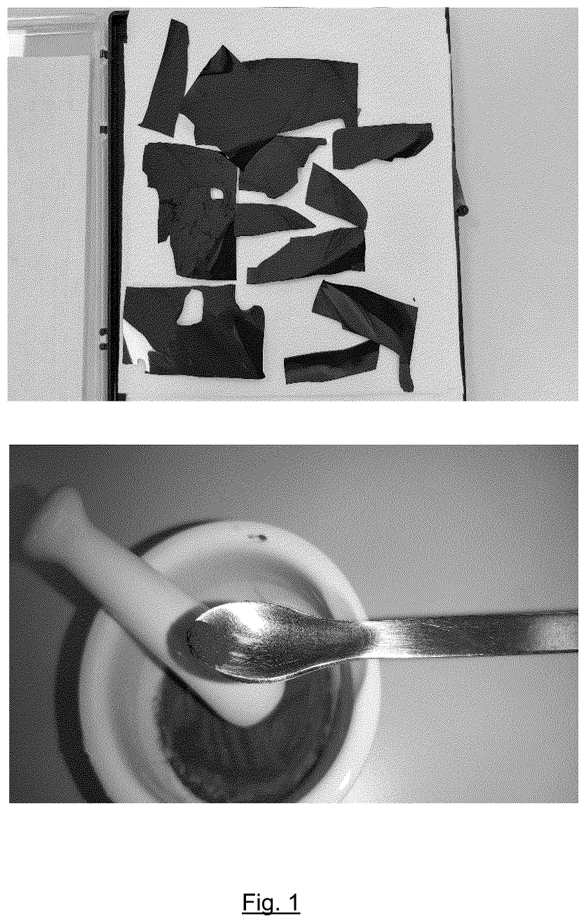

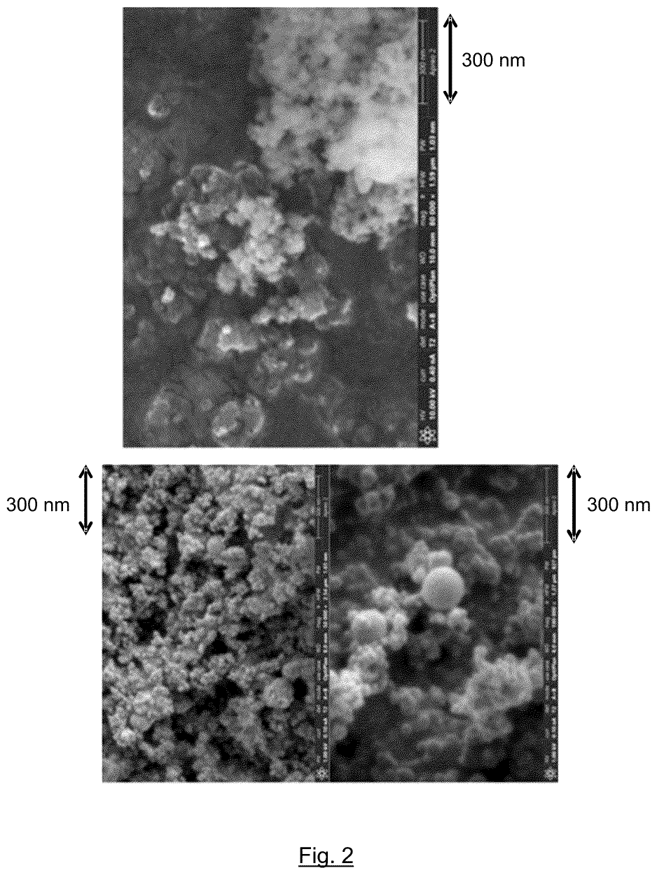

The manufacture of doped-silicon is well understood in the semiconductor industry and includes ion implantation and diffusion methods. Doped silicon is therefore readily available. Optionally, silicon can be doped by using a diffusion method to increase the amount of dopant present in the silicon. As an example of a diffusion method, silicon powder and a doping reagent (for example B₂O₃ for boron doping) are placed in a bowl, which is mixed and placed under an N₂ atmosphere and subjected to a temperature of between 1050° C. and 1175° C. for a few minutes to allow the dopant (for example boron) to diffuse into the silicon. FIGS. 1 and 2 show boron doped silicon produced by this method.

According to certain embodiments, doping of the silicon is heavy. Heavy boron doping is especially preferred. Heavy doping is understood to mean doping of at least 1×10¹⁵ dopant atoms per cm³. In some embodiments dopant is present at levels of at least 1×10¹⁶ dopant atoms per cm³, at least 1×10¹⁷ dopant atoms per cm³, at least 1×10¹⁸ dopant atoms per cm³, at least 1×10¹⁹ dopant atoms per cm³, or at least 1×10²⁰ dopant atoms per cm³.

When boron is used as the dopant, as is preferred, doping levels of 1×10¹⁵ dopant atoms per cm³, and 1×10²⁰ dopant atoms per cm³ correspond, respectively, to a resistivity of 20 mohm-cm, and 1 mohm-cm. The various aspects of the invention in which boron is the preferred dopant, do not exclude silicon which in addition to being doped, for example heavily doped, with boron, is also doped with other elements. According to preferred embodiments of all aspects of the invention, the majority dopant is boron.

Ratio of Silicon to mRNA

Preferably, the ratio by weight of silicon to mRNA is between 0.01:1 and 1:16, such as between 0.01:1 and 1:8; between 1:1 and 1:6, between 1:1 and 1:5, between 1:1 and 1:4, or between 1:1 and 1:3. Preferably, the ratio by weight of silicon to mRNA is between 1:1 and 1:3. Such ratios by weight of silicon to mRNA may further optimise binding of the mRNA to the particles and may thus stabilise the mRNA conveyed by the particles; optionally, such ratios may affect the rate of release of the mRNA, such as at a target treatment site in vivo.

Proposed Non-Limiting Mechanism for mRNA Stabilisation (by Association with Silicon Surface)

At least 70% by weight, for example at least 80% by weight, for example at least 90% by weight of the mRNA present may be associated with the one or more particles comprising hydrolysable silicon. By “associated with”, it may be meant that the mRNA is electrostatically bound to the one or more particles comprising hydrolysable silicon.

It is thought that, when this takes place, the Brownian motion of the mRNA may decrease, such that opportunities for the mRNA to be degraded are further reduced.

Additionally or alternatively, the binding to silicon may (e.g., sterically) prevent endonucleases from orientating to achieve binding and cleavage of the mRNA.

The sequestering of the mRNA away from water molecules, by binding to the silicon, may reduce the activity of enzymes to degrade the mRNA for the reason that water is required for the hydrolysis reactions involved in degradation to occur.

This is in contrast to conventional liposomes, in which mRNA may be relatively free to move and may be accessible to enzymes, as well as being surrounded by water molecules (such as those trapped within a liposomal compartment; or those outside a liposome).

Advantageously, the surface charge (as evidenced by zeta potential, especially a zeta potential of large modulus, such as a modulus of at least about 30, 40 or 50; especially, a large positive zeta potential, of at least about +30, +40 or +50) of the particles comprising hydrolysable silicon may be modulated by the one or more lipids. This may facilitate Si-mRNA binding. As described herein (and as evidenced by the Examples below), the modulation of surface charge may especially occur upon using one or more ionisable lipids; and/or one or more PEGylated lipids (wherein polyethylene glycol, i.e. PEG, is part of the molecule).

Modulation of the surface charge may assist the silicon particles to electrostatically bind the mRNA (which typically has a net negative charge, in view of its phosphate backbone). Doping of the silicon (especially, boron doping), the inclusion of amino acid (especially, glycine) and/or the inclusion of trehalose may further modulate the particles' surface charge, enhancing binding of the mRNA; and thus stabilising the mRNA, for example protecting it from degradation by one or more enzymes.

Without wishing to be bound by theory, therefore, it is thought that the particles' surface may attract the mRNA, modulated by the presence of lipid. Additionally, if the silicon is p-doped (especially, boron doped), the mRNA may be attracted (by virtue of its negative charge) to areas of positive charge induced by the p-doping. Once bound at the particles' surface, the steric hindrance of surrounding molecules (such as the one or more lipids; amino acid(s); and trehalose) may protect the mRNA against degradation, whether simple degradation (e.g., due to pH and/or temperature factors) or degradation involving enzymes. Enzymatic degradation typically requires molecular movement and RNA accessibility, which may be reduced or prevented by binding of the mRNA as presently described.

The optional association of the mRNA with the one or more particles comprising hydrolysable silicon may be correlated with, and/or governed by, the hydrolysis of the silicon. Thus, the rate at which the mRNA becomes bioavailable in vivo may be correlated with the hydrolysis of the silicon, thereby avoiding dose-dumping and/or ensuring gradual release of the mRNA over a suitably long period of time. This may optimise the pharmacokinetic half-life of the mRNA. It may optimise the duration of mRNA bioavailability, as evidenced by Examples 4 to 6 herein showing adequate transfection with chemically unmodified mRNA (Example 4) and stability in serum (Example 5) even at 37° C. (Example 6).

### Lipids

It is especially preferred that the one or more lipids are or comprise (i) one or more ionisable lipids; and/or (ii) one or more PEGylated lipids. Thus, suitably, the one or more lipids is or ae selected from one or more of DOPE, stearylamine, DC-Chol, DOTAP, DSPE-PEG2000 and derivatives thereof, in accordance with the Examples below. Nevertheless, the lipid may comprise others, based on the common behaviour (especially, charge behaviour, and impact on zeta potential) of lipids.

The term “ionisable lipid” is well known in the art. As used herein, therefore, it may refer to lipids with a group capable of becoming positively charged (typically, having a Lewis base (hydrogen acceptor) head group attached, e.g. via some spacer, to a hydrophobic tail). Ionisable lipids typically include a head portion containing at least one tertiary amine moiety. Ionisable lipids may be neutral at physiological pH but may become cationic at lower pH, e.g. below pH 6.5, such as the pH found inside a vacuole as part of endosomal escape.

The type of lipid or types of lipids used may affect the rate of degradation of the one or more silicon particles in vivo. For example, one or more lipid molecules may associate non-covalently with a surface or surfaces of the one or more silicon particles; this may be expressed as surface treating the one or more silicon particles with a lipid. For example, the presence of the at least one lipid has been found to allow for the rate of hydrolysis of the silicon to be controlled, such that the silicon hydrolyses to the bioavailable orthosilicic acid (OSA) degradation product rather than insoluble polymeric hydrolysis products. In particular, surface treating a silicon particle with a lipid has been found to have a beneficial effect on the surface charge of silicon particles, providing them with the requisite zeta potential to allow for improved stabilisation of the mRNA and optionally controlling the rate of release of the nucleic acid at a target site.

Some conventional liposome-based mRNA delivery vehicles may display cytotoxicity. Commonly used systems rely on exogeneous cationic lipid(s) (i.e., lipid(s) which has or have a net positive charge at a pH of about 7.4) containing multiple amine groups. Although well-suited to the electrostatic loading of polyanionic mRNA, these amine-rich species may lead to cytotoxicity, immunogenicity, and non-specific tissue accumulation. Similar problems occur with other, polymer-based, amine-rich delivery systems.

In contrast, the one or more lipids according to the present disclosure preferably exclude toxic lipids. The one or more lipids may, especially, exclude toxic exogenous lipids having a positive charge at physiological pH (known in the art as exotic cationic lipids); such lipids may have an unfavourable toxicity profile, for example by eliciting an innate immune response upon administration to a human patient, especially when they are amine-rich. Although well-suited to the electrostatic loading of polyanionic nucleic acids (including various form of RNA) these (amine-rich) exotic lipid species may lead to cytotoxicity, immunogenicity, and may even lead, contrary to what is desired, to non-specific tissue accumulation, thus counteracting the aim of targeted delivery. Furthermore, they may be complex to synthesise and may therefore be expensive. Thus, preferably, the one or more lipids according to the present disclosure are configured or selected to not elicit an immune response upon administration of the described mRNA vector composition to a human patient in need thereof.

Thus, the one or more lipids in accordance with the present disclosure may exclude amine-rich lipid. Amine-rich lipids may be defined as having more than 2, 3 or 4 nitrogen atoms per molecule of lipid. In sharp contrast, the one or more lipids in accordance with the present disclosure preferably contain up to 1 nitrogen atom per molecule of lipid.

Moreover, it has been found that the beneficial effects disclosed herein may be achieved without necessarily being tied to one or more specific lipid compounds. The one or more lipids may play a role in charge-charge interactions, such as in modulating the zeta potential at the surface of silicon particles, so as to enable better binding of the mRNA. This effect may be seen across a wide range of lipids. Lipids as a class show trends in properties, especially in terms of inter-molecular interactions, enabling the compositions of the present disclosure to be implemented with different lipids and in differing amounts, compared to the specific lipids disclosed in the Examples below.

Ester hydrolysis of the lipid may be reduced by silicon-lipid binding, in turn facilitating lipid-assisted stabilization of the mRNA.

The lipid molecules may bind to one or more of the silicon particles, resulting in a stabilised complex. Positively charged species such as cationic lipids (and other lipid components bearing a positive charge, including, but not limited to, phospholipids) may bind to one or more particles comprising hydrolysable silicon, thereby stabilising these positively charged components.

The lipid or lipids may include one or more of: phosphatidylcholine (PC), hydrogenated PC, stearylamine (SA), dioleoylphosphatidylethanolamine (DOPE), cholesteryl 3β-N-(dimethylaminoethyl) carbamate hydrochloride (DC)-cholesterol, 1,2-dioleoyl-3-trimethylammonium-propane (DOTAP) and derivatives of any thereof. In certain embodiments, the lipid comprises or consists of DOTAP. The type of lipid used to treat the surface of the nanoparticle may affect the stabilisation, optionally the rate of release, of the mRNA. In particular, where there is an association between lipid molecules and one or more silicon particles, there is a beneficial effect on the surface charge of the silicon particles, providing them with the requisite zeta potential to allow for improved stabilisation of mRNA, and optionally controlling the rate of mRNA release at a target site. The presence of the at least one lipid may allow for the rate of hydrolysis of the silicon to be controlled, such that the silicon hydrolyses to the bioavailable orthosilicic acid (OSA) degradation product rather than insoluble polymeric hydrolysis products. Controlling the rate of hydrolysis of the silicon will affect how long the protection of the mRNA is sustained for.

The lipid or lipids may have an average molecular weight in the range of from 500 to 1000 (for example, when the lipid contains one or more of a cationic lipid (for example, DTDTMA (ditetradecyl trimethyl ammonium), DOTMA (2,3-dioleyloxypropyl-1-trimentyl ammonium), DHDTMA (dihexadecyl trimethyl ammonium)) DOTAP, a helper lipid, a structural lipid and a PEG lipid, or is selected from one or more of PC, hydrogenated PC, SA, DOPE, DOTAP, DTDTMA, DHDTMA, DC-cholesterol, and derivatives thereof) the ratio of lipid (i.e. total lipid components) to silicon, before any extrusion or filtration process takes place, is between 1:1 and 20:1, for example between 1:1 and 18:1, 1:1 and 16:1, 1:1 and 11:1, 1:1 and 10:1, 1:1 and 9:1, 1:1 and 8:1, 1:1 and 13:1, 2:1 and 12:1, 2:1 and 11:1, 2:1 and 10:1, 2:1 and 9:1, 2:1 and 8:1, for example between 1:1 and 7:1, between 2:1 and 7:1, between 3:1 and 6:1, between 4:1 and 5:1. Without wishing to be bound by theory, this ratio of lipid to silicon may optionally provide a vesicle system able to control the release of, and stabilise mRNA in contact with the particle of hydrolysable silicon and to facilitate the controlled release of the bioavailable degradation product of the silicon, OSA.

Advantageously, the lipid exerts a significant effect on the surface charge of the silicon particles, which may be measured by reference to zeta potential. Additionally or alternative, using doped silicon (for example p-doped silicon, for example boron-doped silicon as described herein) may further modulate the zeta potential. Modulating the surface charge of the silicon particles in this way may control their ability to remove or sequester water molecules and thus protect the mRNA from degradation. Preferably, the modulus of the zeta potential is increased by the presence of the lipid (and/or by doping the silicon, such as by boron doping).

The lipid or lipids component may be or comprise a phospholipid. The term “phospholipid” refers to a lipid comprising a fatty acid chain and a phosphate group. Phospholipids are typically neutral molecules in that they do not have an overall charge or may carry a negative charge, unlike a cationic lipid which is positively charged. Phospholipids are typically zwitterionic compounds comprising both positive and negatively charged components, but no overall charge. As such, phospholipids are typically classified as neutral lipids. Particularly suitable phospholipids are glycerophospholipids; those in which the polar head group is linked to quaternary ammonium moieties, such as phosphatidylcholine (PC) or hydrogenated phosphatidylcholine; DOPE (phosphatidyl ethanolamine or 1,2-dioleoyl-sn-glycero-3-phosphoethanolamine); and phospholipids which are or are derived from lecithin.

The lipid or lipids component may be or comprise a PEG lipid. Lipids with ether side chains may be referred to as “PEG lipids” or “PEGylated” lipids. The PEG-lipid may be a phospholipid such as 1,2-distearoyl-sn-glycero-3-phosphoethanolamine (DSPE) with a PEG side chain, e.g. DSPE-mPEG2000.

Use of a p-dopant, in accordance with certain preferred embodiments, such as boron (which is preferred) will modulate the silicon particles' zeta potential, typically increasing its modulus and/or making the zeta potential more positive (i.e., less negative). It can therefore be understood that boron doped silicon can more easily achieve a zeta potential of a large modulus and/or a positive zeta potential when treated with a cationic lipid. This means that a positive surface zeta potential may be achieved with a lower amount of cationic lipid or with a wider range of cationic lipids, when doped silicon particles are used.

It also means that even if the cationic lipid degrades during storage, the zeta potential of the silicon particles will remain in a suitable range for longer.

It has been found that lipid to boron-doped silicon molar ratios of between 0.8:1 and 20:1 are particularly advantageous, for example 1:1, 6:1, 8:1, or 10:1, or 12:1 or 16:1.

The lipid or lipids component may, in some embodiments, be or comprise a cationic lipid. The term “cationic lipid” refers to lipid which is positively charged at physiological pH; typically, comprising or consisting of positively charged molecules having a cationic head group attached via a suitable spacer to a hydrophobic tail. Examples include DTDTMA (ditetradecyl trimethyl ammonium), DOTMA (2,3-dioleyloxypropyl-1-trimentyl ammonium), DOTAP, DHDTMA (dihexadecyl trimethyl ammonium) and stearylamine (SA). The positive charge is typically stabilised by a negative counter ion. In preferred embodiments, especially in relation to vaccine compositions, the cationic lipid is, or comprises DOTAP.

The lipid or lipids may comprise phosphatidylcholine, hydrogenated phosphatidylcholine, stearylamine, and/or combinations thereof. In certain embodiments the lipid may consist substantially of phosphatidylcholine, hydrogenated phosphatidylcholine, stearylamine, or combinations thereof.

In certain embodiments the lipid may consist of at least 5% by weight of hydrogenated phosphatidylcholine, for example at least 20 wt %, typically at least 30 wt % and especially at least 50 wt % hydrogenated phosphatidylcholine based on the total weight of the particle. It has been found that hydrogenated phosphatidylcholine to silicon molar ratios of between 0.8:1 to 5:1 are particularly advantageous, for example 1:1, 1.5:1, 2:1, 2.5:1, 3:1, 3.5:1, 4:1, or 4.5:1.

In certain embodiments the lipid may consist of at least 5% by weight phosphatidylcholine, for example at least 20 wt %, typically at least 30 wt % and especially at least 50 wt % phosphatidylcholine, based on the total weight of the particle. It has been found that phosphatidylcholine to silicon molar ratios of between 0.8:1 to 5:1 are particularly advantageous, for example 1:1, 1.5:1, 2:1, 2.5:1, 3:1, 3.5:1, 4:1, or 4.5:1.

In certain embodiments the lipid may consist of at least 5% by weight of stearylamine, for example at least 20 wt %, typically at least 30 wt % and especially at least 50 wt % stearylamine based on the total weight of the particle. It has been found that stearylamine to silicon molar ratios of between 0.8:1 to 5:1 are particularly advantageous, for example 1:1, 1.5:1, 2:1, 2.5:1, 3:1, 3.5:1, 4:1, or 4.5:1. In certain embodiments the lipid may consist of PC and SA, preferably in a ratio by weight of PC:SA of from 1:1 to 20:1, more preferably 7:1 to 10:1, such as a ratio by weight of PC:SA of 72:8. In certain embodiments the lipid may consist of DOPE, SA, and DC-cholesterol.

The ratio by weight of DOPE:SA may be in a range of from 1:1 to 10:1, for example from 4:1 to 8:1. The ratio by weight of DOPE: DC-cholesterol may be in a range of from 1:1 to 5:1, for example from 1:1 to 3:1. The ratio by weight of SA:DC-cholesterol may be in a range of from 1:1 to 1:5, for example from 1:2 to 1:4. In some embodiments, the ratio by weight of DOPE:SA:DC-cholesterol may be 48:8:24.

In certain preferred embodiments the lipid may consist of DOTAP, DOPE and a PEG-lipid (such as mPEG2000-DSPE).

The weight ratio of DOTAP:DOPE may be from 1:2 to 2:1, for example approximately 1:1. The ratio of DOTAP:PEG-lipid and DOPE:PEG-lipid may be 10:1 to 5:1, for example approximately 7:1. The total weight of ration of total lipid to silicon may be between 20:1 and 10:1, for example approximately 16:1.

Polycationic mRNA-Binding Component

The compositions of the invention optionally further comprise a polycationic mRNA binding component. The term “polycationic mRNA-binding component” is well known in the art and refers to polymers having at least 3 repeat cationic amino acid residues or other cationic unit bearing positively charged groups, such polymers being capable of complexion with a mRNA under physiological conditions. An example of a mRNA-binding polycationic molecule is an oligopeptide comprising one or more cationic amino acids. Such an oligopeptide may, for example, be an oligo-lysine molecule, an oligo-histidine molecule, an oligo-arginine molecule, an oligo-ornithine molecule, an oligo diaminopropionic acid molecule, or an oligo-diaminobutyric acid molecule, or a combined oligomer comprising or consisting of any combination of histidine, arginine, lysine, ornithine diaminopropionic acid, and diaminobutyric acid residues. Further examples of polycationic components include dendrimers and polyethylenimine.

### Amino Acids

All aspects of the invention may include the additional optional presence of one or more amino acids; especially arginine, tyrosine and/or glycine, most especially glycine.

In its broadest sense, the term “amino acid” encompasses any artificial or naturally occurring organic compound containing an amine (—NH₂) and carboxyl (—COOH) functional group. It includes an α, β, γ and δ amino acid. It includes an amino acid in any chiral configuration. According to some embodiments (for example, when the silicon-containing particles of the invention are formulated with one or more of PC, hydrogenated PC, SA, DOPE, DOTAP, DC-cholesterol, and derivatives thereof) the amino acid is preferably a naturally occurring a amino acid. It may be a proteinogenic amino acid or a non-proteinogenic amino acid (such as carnitine, levothyroxine, hydroxyproline, ornithine or citrulline). In preferred embodiments the amino acid comprises arginine, histidine, or glycine, or a mixture of arginine and glycine. In particularly preferred embodiments, the amino acid comprises glycine. Such amino acids may function to stabilise the silicon particle and control the hydrolysis of silicon both in storage and in vivo. This dual action of aminoacids i.e. interact with both silicon and nucleic acid through electrostatic interaction, hydrogen bonds and Vanderwals interaction as well as water-mediated interactions. Depends on the type of amino acids, the ratio between silicon:nucleic acid, & silicon surface area, the complex generates different pH environment. It is therefore plausible to say that the interaction between which side of amino acids, i.e. carboxy or amine group with silicon are modulated by charge screening as a function of electrolyte concentration. (see J. Phys. Chem. B 2013, 117, 10742-10749)

Treating the lipid-treated silicon particles with an amino acid may also provide a beneficial stabilising effect on the nucleic acid. Treating lipid-treated silicon particles with amino acids has been shown to stabilise nucleic acids in biological fluids, for example in ocular tissues and plasma and tissue fluid. Lipid-treated particles formulated with an amino acid in this manner may be particularly suitable for delivery to the body, for example delivery by transcutaneous injection.

### Peptide

In addition to the amino acids described above, in all aspects of the invention there may be included a peptide containing a cell surface receptor-, for example integrin-, recognition sequence that confers a degree of cell specificity to the particle. The peptide may have a “head group” containing a cell surface receptor recognition sequence and additionally a “tail” that can bind non-covalently to the mRNA and/or bind to the silicon.

Which side-chains exhibits more favourable interactions again depends on various factors including the pH of complex and silicon surface state. In general, at lower pH with predominantly neutral silanol surface groups, it appears that the glycine carboxylate group interacts with the surface and that the amine group extends into solution. However, as the pH increases and the silicon surface becomes negatively charged, the positively charged amino moiety of glycine interacts more favourably with the surface, and the negatively charged carboxylate group becomes exposed. Arginine binds to silicon surfaces in a pH-dependent manner, with increased affinity at higher pH, presumably mediated by favourable electrostatic interactions between the positively charged side chains and negatively charged silica surface

### Ratio of Amino Acid(s) to Silicon

Preferably, when amino acid(s) is/are present, the ratio of amino acid to silicon is between 0.05:1 and 2:1, for example between 0.05:1 and 1.8:1, 0.05:1 and 1.6:1, 0.05:1 and 1.4:1, 0.05:1 and 1.2:1, 0.05:1 and 1:1, 0.05:1 and 0.9:1, 0.05:1 and 0.8:1, 0.05:1 and 0.6:1, 0.05:1 and 0.5:1, 0.05:1 and 0.4:1, 0.05:1 and 0.3:1, 0.05:1 and 0.2:1, preferably between 0.2:1 and 0.8:1, especially between 0.3:1 and 0.7:1. The ratio of amino acid to silicon may be between 0.05:1 to 0.4:1, for example between 0.08:1 and 0.35:1, especially 0.09:1 to 0.32:1. Advantageously, tuning the ratio of amino acid to silicon further stabilises the mRNA.

In some embodiments, the amino acid is a combination of arginine and glycine, wherein the ratio of Arg:Gly is between 1:0.6 and 3:1, for example between 1:0.8 and 2.5:1, for example between 1:1 and 2:1.

According to other embodiments of all aspects of the invention the particles are formulated with arginine. Preferably, the ratio of arginine to silicon is between 0.05:1 to 0.4:1, for example between 0.08:1 and 0.35:1, especially 0.09:1 to 0.32:1.

According to other embodiments of all aspects of the invention the particles are formulated with glycine. Preferably, the ratio of glycine to silicon is between 0.05:1 to 0.5:1, for example between 0.08:1 and 0.45:1, especially 0.09:1 to 0.42:1.

Preferred amino acids for use with all aspects of the invention include arginine, glycine, proline, lysine and histidine and mixtures of two or more thereof; especially, arginine, tyrosine and/or glycine; most especially glycine.

### Non-Reducing Disaccharide

Optionally included in the described composition is at least one non-reducing disaccharide. The non-reducing disaccharide may optionally be selected from sucrose, trehalose, raffinose, stachyose and verbascose or mixtures of any thereof, most preferably the non-reducing disaccharide is trehalose, or a mixture comprising trehalose.

The non-reducing disaccharide (for example, trehalose or a mixture comprising trehalose) is optionally present at a weight ratio to silicon of at least 1:1000, at least 1:100, at least 1:50, at least 1:10, at least 1:1, or at least 1:0.5. Preferably, the non-reducing disaccharide is trehalose which is optionally present a weight ratio to silicon of at least 1:1000, at least 1:100, at least 1:50, at least 1:10, at least 1:1, or at least 1:0.5.

It is postulated that non-reducing disaccharides, especially trehalose, may act as a desiccation-protectant. Non-reducing disaccharides, especially trehalose, may sequester water molecules away from the mRNA, thus further reducing the rate of degradation of the mRNA by hydrolysis.

Additionally or alternatively, the non-reducing disaccharide may trap mRNA molecules, thereby restricting their movement; this may assist the trapping effect of the silicon particles, as described herein. Trehalose may be particularly effective at trapping water molecules, which is of value to prevent mRNA degradation. Thus, the water-sequestering effect of the silicon particles may be enhanced.

An additional advantage of including a non-reducing disaccharide, such as trehalose, is that the presence of the non-reducing disaccharide may facilitate re-suspension of a pharmaceutical composition from a powdered material. Thus, the formulation may be easier to handle and to process for delivery to patients in need thereof.

### Especially Preferred Combinations

According to all aspects of the invention, boron doping (especially heavy boron doping as defined above) is preferred.

It is preferred that the mRNA is or comprises an mRNA vaccine, encoding an antigen.

The following features may especially be preferred, especially in combination: It is especially preferred that the mRNA is or comprises mRNA wherein at least about 80% of the ribonucleosides of the open reading frame of the mRNA have the same molecular structure as naturally occurring ribonucleosides; and/or the mRNA has no non-naturally occurring secondary structure.

It is especially preferred that the lipid is or comprises an ionisable lipid, for example one or more cationic lipids, such as DOTAP, and/or one or more zwitterionic lipids, such as one or more phospholipids, such as DOPE; or both (for example, wherein the lipid comprises DOTAP or wherein the lipid comprises both an ionisable lipid and DOTAP).

It is more especially preferred that the one or more lipids are or comprise (i) one or more ionisable lipids; and/or (ii) one or more PEGylated lipids. Thus, suitably, the one or more lipids is or ae selected from one or more of DOPE, stearylamine, DC-Chol, DOTAP, DSPE-PEG2000 and derivatives thereof, in accordance with the Examples below. Nevertheless, the lipid may comprise others, based on the common behaviour (especially, charge behaviour, and impact on zeta potential) of lipids.

It is especially preferred to include glycine in the composition, in accordance with the Examples below.

It is especially preferred to include trehalose in the composition, in accordance with the Examples below.

Upon formulation, it is preferred that the composition is embedded in a hydrogel, especially a sodium hyaluronate hydrogel, such as that of Example 4 below.

### Pharmaceutical Compositions

According to the invention, there is provided a pharmaceutical composition comprising the mRNA vector composition of the first aspect of the invention, wherein the pharmaceutical composition is a vaccine composition.

In some embodiments, mRNA has a half-life in the pharmaceutical composition at 4° C. of at least 3 months, for example at least 4, 5 or 6 months.

In some embodiments, pharmaceutical and mRNA vector compositions of the invention are in a form suitable for intramuscular injection.

Pharmaceutical and vector compositions of the invention may comprise excipients, including but not limited to: preservatives, cryoprotectants and immune adjuvants. As used herein, the term adjuvant may refer to a substance that modulates, for example increases, the immune response of a subject to the vaccine composition. By way of non-limiting example, an adjuvant may be or may comprise one or more lipids, proteins, CpG oligodeoxynucleotides, (which may be part of the final product as an excipient or incorporated into the lipid particles along with the mRNA, or both) and/or other molecular adjuvants. The pharmaceutical and vector compositions may in some embodiments comprise one or more buffer components. The pharmaceutical and mRNA vector compositions may in some embodiments comprise trometamol. The pharmaceutical and mRNA vector compositions may in some embodiments comprise trometamol hydrochloride. The pharmaceutical and mRNA vector compositions may in some embodiments comprise acetic acid. The pharmaceutical and mRNA vector compositions may in some embodiments comprise sodium acetate trihydrate. The pharmaceutical and mRNA vector compositions may in some embodiments comprise potassium chloride. The pharmaceutical and mRNA vector compositions may in some embodiments comprise potassium dihydrogen phosphate. The pharmaceutical and mRNA vector compositions may in some embodiments comprise sodium chloride. The pharmaceutical and mRNA vector compositions may in some embodiments comprise disodium hydrogen phosphate dehydrate. The pharmaceutical and mRNA vector compositions may in some embodiments comprise sucrose. It will be appreciated that the pharmaceutical and mRNA vector compositions of the invention may optionally be diluted with saline before administration by intramuscular injection. In some embodiments the pharmaceutical and mRNA vector compositions may be a powder. In some embodiments the pharmaceutical and mRNA vector compositions may comprise a liquid.

In the pharmaceutical and mRNA vector composition of the invention, the mRNA can be protected from enzymatic degradation by the one or more particles comprising hydrolysable silicon, which sequester or remove water as described herein.

### Preparation of Particles Comprising Hydrolysable Silicon

The particles comprising hydrolysable for use in the invention may conveniently be prepared by techniques conventional in the art, for example by milling processes or by other known techniques for particle size reduction.

The silicon-containing particles may be made from sodium silicate particles, colloidal silica or silicon wafer materials, such as the silicon wafer described in Example 1 hereinbelow. Macro, micro or nano scale particles may be ground in a ball mill, a planetary ball mill, or other size reducing mechanism.

The resulting particles may be air classified or sieved to recover particles of a uniform required size. It is also possible to use plasma methods and laser ablation for the production of particles.

Porous particles may be prepared by methods conventional in the art, including the methods described herein.

Thus, it will be appreciated that the particles may be produced by various techniques familiar to the skilled person.

The techniques may include, for example, those described in Example 1 hereinbelow.

The techniques may include physical (sometimes referred to, in the art, as “non-wet”) processes having bulk silicon (especially, silicon wafer) as the starting material; such as pulsed laser ablation, thermal degradation and ball milling. Thus, the particles may be obtainable by a method comprising or consisting of one or more of pulsed laser ablation, thermal degradation and ball milling, of bulk silicon (especially, silicon wafer).

Additionally or alternatively, the particles may be produced by chemical (sometimes referred to, in the art, as “wet”) techniques, including but not limited to electrochemical etching of bulk silicon (especially, silicon wafer). Such techniques optionally include the HF etching described above. Thus, the particles may be obtainable by a method comprising or consisting of electrochemical etching of bulk silicon (especially, silicon wafer).

Once formed, silicon particles may be sorted by size, such, for example, as by air classification, sieving and/or filtration. Thus, the particles may be obtainable by a method comprising one or more of air classification, sieving and/or filtration.

Thus, for example, the particles may be obtainable by a method comprising producing silicon particles from bulk silicon, especially from silicon wafer, such as by one or more of pulsed laser ablation, thermal degradation, ball milling, and electrochemical etching, of bulk silicon (especially, silicon wafer); followed, optionally, by sorting by size, such as by air classification, sieving and/or filtration. Optionally, the particles may be washed before use, such as in methanol or ethanol, to remove a thin oxidised layer from their surface. In the art, this may be termed “activation”.

The particles thus obtained may have a narrow size distribution and homogeneous surface chemistry, leading to batch-to-batch reliability and reproducibility of one or more of the advantages described herein.

Suitable physical and chemical techniques are set out, for example, in WO 2011/012867 A1 (in the name of SISAF LTD); in Tokarska K et al., Facile production of ultra-fine silicon nanoparticles, R. Soc. Open Sci., 2020, 7: 200736; and in Kim, T., Lee, J. Silicon nanoparticles: fabrication, characterization, application and perspectives, Micro and Nano Syst. Lett, 2023, 11: 18, each of which is incorporated herein by reference in its entirety.

Preparation of Pharmaceutical and mRNA Vector Compositions

Pharmaceutical compositions and mRNA vector compositions of the invention may be produced by bringing together the components. In some embodiments, this bringing together may merely involve mixing solutions of the components, for example under conditions leading to their complexation. The bringing together may involve contacting the mRNA with the particles of silicon containing material prior to the addition of the lipid components. Addition of the lipid components after contact of the mRNA with the silicon containing particles may facilitate the formation of a lipid shell as described herein, encapsulating a core, the core comprising both the one or more particles and the mRNA.

It has been found to be advantageous, and is therefore preferred, that surface oxidation of particles comprising hydrolysable silicon is removed (i.e., the particles are “activated”), before being bought into contact with other components of the invention. This may be carried out by dispersing the particles (e.g. porous doped-silicon nanoparticles) in a volatile alcohol or other volatile solvent (such as chloroform, methanol, ethanol or propanol, e.g. methanol) prior to bringing them into contact with other components of the invention.

If the non-reducing disaccharide (e.g., trehalose) and/or amino acid (e.g., glycine) is or are present, the silicon particles may be mixed with the non-reducing disaccharide and/or amino acid.

The silicon particles are then contacted with the one or more lipids. Preferably, the particles and lipid(s) are then extruded together.

The mRNA is then added.

### Lyophilisation and Preparation of the Composition in a Delivery Device

Optionally, the mRNA vector composition is lyophilised, optionally lyophilised with a cryoprotectant and/or a lyoprotectant, such as one or more sugars, such as sucrose and/or trehalose, for example to form a powder. Lyophilisation removes water and enables a dry powder to be produced.

Such a dry powder may optionally be dispersed, for example, in a hydrogel.

Other physical forms of the compositions of the invention include liquid forms and frozen forms.

In some embodiments, the composition is provided in a delivery device, for example in an injection device such as a syringe or a multiplicity of microneedles.

### Particle Configuration and Structural Features of Described Compositions (for Promoting Stabilisation)

Preferably, the particles comprising hydrolysable silicon are complexed with the one or more lipids thus forming a delivery vehicle for transport of the mRNA. Thus, when the mRNA is added, it also becomes complexed with the particles and/or the lipid. Put another way, the particles and lipid are organised into a delivery vehicle that is loaded with the mRNA. Advantageously, this may make the mRNA less liable to react with one or more external reactive species. The mRNA may be less at risk of degradation catalysed by enzymes external to the complex, especially in vivo, such as during circulation in the body and/or in a cell cytoplasm.

In its broadest sense, as used here, the term “complexed with” may encompass ionic and/or covalent and/or physical interactions, and especially may encompass charge-charge interactions, such as those resulting from the particles' zeta potential.

Thus, preferably, the zeta potential of the particles, especially when modulated by the one or more lipids and any other components present, is such as to attract and facilitate binding of the mRNA.

In preferred embodiments wherein amino acid(s) is or are present, the amino acid(s) may also complex with the particles, lipid(s) and/or mRNA. The amino acid(s), especially when charged, may modulate the particles' zeta potential thus modulating mRNA and/or lipid complexation with the particles.

When other advantageous components are present, they may also complex with the particles, lipid(s), amino acid(s) and/or mRNA.

Electrostatic phosphate-silanol interactions may play a role in the mechanism of nucleic acid binding to silicon nanoparticles. These and other short-range Si-nucleic acid attractions may be sufficiently strong to overcome any (more delocalised) electrostatic repulsion between the mRNA (having a net negative charge) and the silicon nanoparticle surface. Phosphate-silanol interactions may occur where the oxygen of a phosphate group of the RNA backbone closely approaches the hydrogen of a surface silanol group. Meanwhile, complementary hydrophobic interactions may occur when a nucleobase lies against a silanol-free region of the silicon nanoparticle surface.

Thus, mRNA is preferably bound to the surface of the silicon particles by electrostatic phosphate-silanol interactions; and/or nucleobase-silicon attraction.

### Lipidic Structures

The one or more lipids may be formed of, or may comprise, one or more lipidic structures. Said structures may be or comprise one or more of: micelles, incomplete micelles, liposomes, incomplete liposomes and (e.g., solid or semi-solid) lipid globules, as described herein.

Meanwhile, preferably, the mRNA is bound (especially, bound non-covalently) to the particles comprising hydrolysable silicon. Preferably, at least about 50, 60 or 70% of mRNA in the composition is bound to the particles in this way.

In turn, preferably, the particles comprising hydrolysable silicon are bound to the surface of, and/or are present in the interior of, the one or more lipidic structures.

Thus, preferably, particles comprising hydrolysable silicon are bound to the surface of, and/or are present in the interior of, one or more of: micelles, incomplete micelles, liposomes, incomplete liposomes, and (e.g., solid or semi-solid) lipid globules; and mRNA is bound to the particles comprising hydrolysable silicon. Preferably, the particles comprising hydrolysable silicon are present in one or more aggregates of particles comprising hydrolysable silicon, especially one or more aggregates comprising or consisting of chains of the particles, most especially chains that extend into the interior of the one or more lipidic structures, such as into the interior of one or more of: micelles, incomplete micelles, liposomes, incomplete liposomes, and (e.g., solid or semi-solid) lipid globules (especially, liposomes and/or lipid globules). An amino acid (especially, glycine, arginine and/or tyrosine, such as glycine) may also be associated with (e.g., bound non-covalently to) the particles comprising hydrolysable silicon; and/or associated with (e.g., on the surface of) the one or more lipidic structures.

As used herein, the term liposomal lipid particle, or the term liposome, may have its normal meaning in the art. Thus, it may refer to a vesicle having at least one lipid bilayer, which may be approximately spherical in shape. A liposome may be visualised as a lipid “bubble” surrounding an interior space. The interior space may be a hydrophilic environment.

The composition may in some embodiments comprise one or more liposomes. I.e., the one or more lipids may be formed of, or may comprise, one or more liposomes.

The composition may in some embodiments comprise incomplete liposomes. In that sense, their interior space may be accessible from the exterior. An incomplete liposome may be visualised as an incomplete lipid “bubble” wherein there are one or more gaps in the (approximately spherical) lipid bilayer surface. Thus, suitably, the one or more lipids may be formed of, or may comprise, one or more incomplete liposomes.

Suitably, the one or more lipids may be formed of, or may comprise, one or more incomplete liposomes and/or one or more (complete) liposomes.

Thus, the present composition may comprise the particles comprising hydrolysable silicon associated with one or more liposomes and/or one or more incomplete liposomes, wherein the mRNA is associated with (especially, bound to) the particles comprising hydrolysable silicon. An amino acid (especially, glycine, arginine and/or tyrosine, such as glycine) may also be associated with the particles comprising hydrolysable silicon.

The optionally present liposomes or incomplete liposomes, may have a mean diameter in a range of from about 50 nm to about 400 nm, especially about 50 nm to about 200 nm, more especially about 60 nm to about 100 nm.

mRNA may be bound non-covalently to particles comprising hydrolysable silicon that are bound to the surface of one or more liposomes and/or one or more incomplete liposomes. Up to about 10 or 20% of the total mRNA present in the composition may be bound non-covalently to particles comprising hydrolysable silicon that are, in turn, bound to the surface of one or more liposomes and/or one or more incomplete liposomes.

mRNA may be bound non-covalently to particles comprising hydrolysable silicon that are, in turn, in the interior of one or more liposomes and/or one or more incomplete liposomes. At least about 50, 60 or 70% of total mRNA present in the composition may preferably be bound non-covalently to particles comprising hydrolysable silicon that are in the interior of one or more liposomes and/or one or more incomplete liposomes.

Preferably, mRNA is bound non-covalently to particles comprising hydrolysable silicon that are in the interior of one or more liposomes and/or one or more incomplete liposomes; and mRNA is bound non-covalently to particles comprising hydrolysable silicon that are bound to the surface of one or more liposomes and/or one or more incomplete liposomes.

In some embodiments, the composition may be free or substantially free of liposomes; and/or may be free or substantially free of incomplete liposomes.

Optionally, the one or more lipids may be formed of, or may comprise, one or more lipid monolayers. Optionally, the one or more lipids may be or comprise one or more micelles or incomplete micelles. It will be understood that micelles have similar characteristics to liposomes, except that micelles' walls are formed of lipid monolayer; whereas liposomes' walls are formed of lipid bilayer. Thus, a micelle may refer to a vesicle having at least one lipid monolayer, which may be approximately spherical in shape. A micelle, similarly to a liposome, may be visualised as a lipid “bubble” surrounding an interior space. The interior space may be a hydrophilic environment.

Thus, the present composition may comprise the particles comprising hydrolysable silicon associated with one or more micelles and/or one or more incomplete micelles, wherein the mRNA is associated with (especially, bound to) the particles comprising hydrolysable silicon.

The optionally present micelles may have a mean diameter in a range of from about 50 nm to about 400 nm, especially about 50 nm to about 200 nm, more especially about 60 nm to about 100 nm.

mRNA may be bound non-covalently to particles comprising hydrolysable silicon that are bound to the surface of one or more micelles and/or one or more incomplete micelles. Up to about 10 or 20% of the total mRNA present in the composition may be bound non-covalently to particles comprising hydrolysable silicon that are, in turn, bound to the surface of one or more micelles and/or one or more incomplete micelles.

mRNA may be bound non-covalently to particles comprising hydrolysable silicon that are, in turn, in the interior of one or more micelles and/or one or more incomplete micelles. At least about 50, 60 or 70% of the total mRNA present in the composition may preferably be bound non-covalently to particles comprising hydrolysable silicon that are in the interior of one or more micelles and/or one or more incomplete micelles.

Preferably, mRNA is bound non-covalently to particles comprising hydrolysable silicon that are in the interior of one or more micelles and/or one or more incomplete micelles; and mRNA is bound non-covalently to particles comprising hydrolysable silicon that are bound to the surface of one or more micelles and/or one or more incomplete micelles.

In some embodiments, the composition may be free or substantially free of micelles; and/or may be free or substantially free of incomplete micelles.

The one or more lipids may be formed of, or may comprise, one or more lipid globules, each globule optionally being surrounded by a layer of surfactants. Lipid globules do not enclose an interior space or cavity. Instead, they are solidly formed, or substantially solidly formed, of lipid, in which other components, such as the particles comprising hydrolysable silicon to which are bound mRNA molecules, may be dispersed. Thus, the globules' interior may be studded with the particles comprising hydrolysable silicon; with mRNA molecules, in turn, being bound (non-covalently) to the particles comprising hydrolysable silicon. Additionally or alternatively (preferably, additionally), the particles comprising hydrolysable silicon, to which are (non-covalently) bound mRNA molecules, may be bound to the surface of one or more lipid globules.

Thus, preferably, the present composition comprises the particles comprising hydrolysable silicon associated with (especially, dispersed within and/or bound onto the surface of) one or more (solid or substantially solid; i.e., non-hollow) lipid globules, wherein the mRNA is associated with (especially, bound to) the particles comprising hydrolysable silicon.

The optionally present lipid globules may have a mean diameter in a range of from about 50 nm to about 400 nm, especially about 50 nm to about 200 nm, more especially about 60 nm to about 100 nm. mRNA may be bound non-covalently to particles comprising hydrolysable silicon that are bound to the surface of one or more lipid globules. Up to about 10 or 20% of total mRNA present in the composition may be bound non-covalently to particles comprising hydrolysable silicon that are, in turn, bound to the surface of one or more lipid globules.

mRNA may be bound non-covalently to particles comprising hydrolysable silicon that are, in turn, in the interior of one or more lipid globules. At least about 50, 60 or 70% of total mRNA present in the composition may preferably be bound non-covalently to particles comprising hydrolysable silicon that are in the interior of one or more lipid globules.

Preferably, mRNA is bound non-covalently to particles comprising hydrolysable silicon that are in the interior of one or more lipid globules; and mRNA is bound non-covalently to particles comprising hydrolysable silicon that are bound to the surface of one or more lipid globules.

### Particle Aggregates

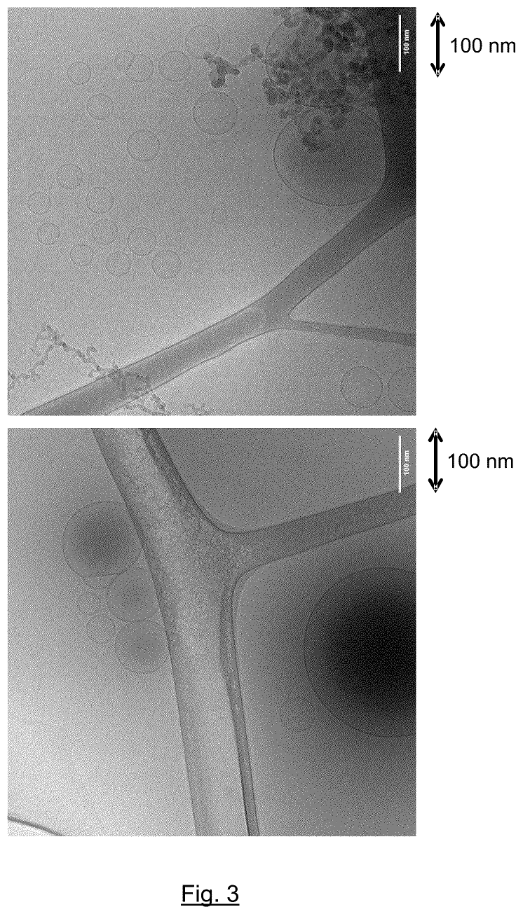

The particles comprising hydrolysable silicon may coalesce into aggregates of particles comprising hydrolysable silicon, for example as shown in the transmission electron microscope (TEM) images of FIGS. 3 to 5.

Thus, the described composition may comprise aggregates (especially, chains) of the particles comprising hydrolysable silicon.

As used herein, the term “aggregate of particles comprising hydrolysable silicon” may refer to a cluster of particles wherein nearest-neighbour particles are in contact with each other. Such clusters may have varying configurations, such as substantially spherical clusters of particles and/or chains of particles. Particularly preferred are configurations comprising or consisting of chains of particles.

Thus, the composition may comprise one or more aggregates of particles comprising hydrolysable silicon. Preferably, the aggregates comprise one or more chains of the particles.

There may, for example, be present at least about 2, 3 or 4 chains on average per aggregate.

The one or more aggregates may comprise one or more branched chains of the particles. Thus, the one or more aggregates may be formed of, or may comprise, branches formed from chains of the particles. There may, for example, be present at least about 2, 3 or 4 branches per aggregate.

The one or more aggregates of particles comprising hydrolysable silicon may be associated with the one or more lipids, for example embedded in lipid and/or attached to the surface of lipid.

The one or more aggregates of particles comprising hydrolysable silicon may be associated with one or more lipidic structures described herein, for example embedded within and/or attached to one or more lipidic structures.

As described herein, the one or more lipidic structures may be formed of or may comprise one or more of: micelles, incomplete micelles, liposomes, incomplete liposomes and lipid globules. Thus, the one or more aggregates of particles comprising hydrolysable silicon may be associated with (for example, embedded in and/or attached to the surface of) one or more of lipid micelles, incomplete lipid micelles, liposomes, incomplete liposomes and lipid globules.

In particular, the one or more aggregates of particles comprising hydrolysable silicon may be embedded in or attached to the surface of one or more of: liposomes; incomplete liposomes; and lipid globules. The ratio of the longest dimension of an aggregate to the longest dimension of a lipidic structure may on average be about 1:5 to 5:1, especially about 1:3 to 3:1; especially when the lipidic structure is or comprises liposomes and/or lipid globules and the one or more aggregates are embedded therein or attached to the surface thereof. This may be measured, for example, by TEM as shown in FIGS. 3 to 5.

The average longest dimension of an aggregate may be about 50 nm to about 500 nm, especially about 50 nm to about 200 nm, such as about 50 to about 150 nm, such as when measured by TEM. In particular, the one or more aggregates may be or comprise one or more chains of particles, with the average length of a chain being about 50 nm to about 500 nm, especially about 50 nm to about 200 nm, such as about 50 to about 150 nm. The average cross-sectional diameter of a chain may be about 5 nm to about 50 nm, such as about 5 nm to about 30 nm.

The ratio of the longest dimension of an individual particle comprising hydrolysable silicon to the longest dimension of a lipidic structure may on average be in a range of from about 1:100 to about 1:2, especially about 1:100 to about 1:5, more especially about 1:100 to about 1:9; especially when the lipidic structure is or comprises liposomes and/or lipid globules and the one or more aggregates are embedded therein or attached to the surface thereof. This may be measured, for example, by TEM as shown in FIGS. 3 to 5.

When the one or more aggregates are present, the average (e.g., mean) diameter of a particle may preferably be about 1 nm to about 50 nm, especially about 1 nm to about 30 nm, more especially about 5 nm to about 20 nm, such as about 10 nm. Additionally or alternatively, the particles may be porous and may have an average (e.g., mean) pore diameter of about 0.1 to about 5 nm, such as about 2 nm.

In turn, mRNA may be bound (non-covalently) to one or more of the particles in the aggregates.

Thus, the one or more aggregates may bind mRNA. When the one or more aggregates are or comprise chains of the particles, such chains may extend into the interior of the lipidic structure(s) (especially, into liposomes, incomplete liposomes, and/or lipid globules). In this way, the chains may provide a route for the mRNA to be better encapsulated into the lipid. Without wishing to be bound by theory, it is thought that this may shield the mRNA from degradation, especially by shielding it from enzymes (especially, in vivo) and preventing or reducing mRNA molecules being available to react with water molecules. It is thought that as the silicon particles degrade over time, mRNA may be released, thus enabling protection of the mRNA until it reaches a target site for release.

The presence of mRNA may itself facilitate aggregation of the particles. mRNA has a negative charge (due to its phosphate backbone), which may induce aggregation of the particles. Additionally or alternatively, the presence of Si—O species on the particles' surface may induce particle-particle interactions to facilitate aggregation.

Thus, preferably, the one or more aggregates are or comprise one or more chains of the particles comprising hydrolysable silicon, wherein mRNA is bound to the particles, and the one or more chains extend into the interior of the lipid structure(s), especially, into liposomes, incomplete liposomes, or lipid globules. This may provide a stabler environment for the mRNA. Additionally or alternatively, it may enable increased mRNA uptake by the lipid structures, compared to no such aggregate(s) being present. This is in contrast to conventional liposomal delivery vehicles, for which inefficient mRNA uptake may be a problem; for example, it is thought that up to about 80% of conventional liposomal delivery vehicles formulated into commercially available therapeutic compositions may be “empty” of mRNA.

Preferably, the one or more lipids do not comprise any structural lipids, such as, especially, cholesterol. Without wishing to be bound by theory, it is thought that the presence of a structural lipid, especially cholesterol, may be unnecessary for the structural stability of lipidic structures when aggregates of particles are present in the described manner.

Optionally, substantially no particles are present as isolated particles; instead, substantially all particles are present in aggregates.

Aggregation may be promoted when the particles comprising hydrolysable silicon have an average diameter of about 1 nm to about 50 nm, especially about 1 nm to about 30 nm, more especially about 5 nm to about 20 nm, such as about 10 nm. Such particle diameters lie below the typical diameter of lipidic structures (as described herein) that may form spontaneously (optionally, promoted by extrusion) from the one or more lipids.

Thus, preferably, the described composition comprises aggregates (especially, chains) of the particles comprising hydrolysable silicon, wherein the particles comprising hydrolysable silicon have an average diameter of about 1 nm to about 50 nm, especially about 1 nm to about 30 nm, more especially about 5 nm to about 20 nm, such as about 10 nm.

Additionally or alternatively, the described composition comprises aggregates (especially, chains) of the particles comprising hydrolysable silicon, wherein the ratio of the longest dimension of an aggregate to the longest dimension of a lipidic structure is on average be about 1:5 to 5:1, especially about 1:3 to 3:1.

Aggregation may be promoted when the composition is extruded, for instance in the manner set out in Example 1 hereinbelow. Extrusion may be or comprise extrusion through a porous membrane having average pore diameters of about 0.01 μm to about 1 μm, such as about 0.05 μm to about 0.6 μm.

Accordingly, the described mRNA vector composition may be (or be formed from) an extruded composition.

Preferably, extrusion occurs before addition of the mRNA. Thus, the composition may be formed from an extruded composition comprising the particles and the one or more lipids, to which mRNA has been added.

Therefore, the described mRNA vector composition may suitably be an extruded composition that comprises aggregates (especially, chains) of the particles comprising hydrolysable silicon, wherein the particles comprising hydrolysable silicon have an average diameter of about 1 nm to about 50 nm, especially about 1 nm to about 30 nm, more especially about 5 nm to about 20 nm, such as about 10 nm.

Similarly, the described method of increasing the stability of a mRNA molecule which is not chemically modified, comprising the step of bringing it into contact with one or more particles comprising hydrolysable silicon and one or more lipids, may first comprise a step of extruding the one or more particles comprising hydrolysable silicon together with one or more lipids, through a porous membrane. The porous membrane optionally has average pore diameters of about 0.01 μm to about 1 μm, such as about 0.05 μm to about 0.6 μm.

### Clauses

Features of the aspects of the invention are set out in the following numbered clauses, not to be confused with the claims.

1. An mRNA vector composition comprising:
mRNA;
one or more particles comprising hydrolysable silicon; and
one or more lipids, characterised in that the mRNA is mRNA which is not chemically modified.

2. An mRNA vector composition according to clause 1, wherein the mRNA is not chemically modified in the sense that:
i. it does not contain any nucleotides that differ from A, U/T, C, and G; and
ii. it does not contain any methylated or acetylated bases (except, optionally, as part of any 5′ cap); and
iii. it does not contain any modified sugar moieties (except, optionally, as part of any 5′ cap).

3. An mRNA vector composition according to clause 1 or clause 2, which is in the form of a lipid nanoparticle.

4. An mRNA vector composition according to any preceding clause, which is a liposomal lipid nanoparticle or a hybrid lipid nanoparticle.

5. An mRNA vector composition according to any preceding clause, wherein the isolated mRNA has a half-life in vivo of under about an hour, especially under about 10 minutes.

6. An mRNA vector according to any preceding clause, wherein the mRNA is of at least about 300 residues.

7. An mRNA vector composition according to clause 6, wherein the mRNA comprises a protein-encoding open reading frame and optionally one or more of:
a 5′cap structure;
a poly(A) tail;
one or more untranslated regions.

8 An mRNA vector composition according to clause 9, wherein the open reading frame of the mRNA encodes an antigen of a pathogen, optionally wherein the open reading frame of the mRNA encodes:
a spike protein antigen of SARS-CoV-2;
a viral protein related to a virus that causes a disease
a bacterial protein related to a disease causing bacterium
a parasitic protein related to a disease causing parasite
a fungal protein related to a disease causing fungal species
a tumour-associated antigen or a tumour-specific antigen;
an allergen; or
a modulator of an immune disease, an autoimmune disease, or an inflammatory disease.

9 An mRNA vector composition according to any preceding clause, further comprising an amino acid, such as glycine; and/or further comprising one or more non-reducing disaccharides, such as trehalose.

10. A method of increasing the stability of a mRNA molecule which is not chemically modified, comprising the step of bringing it into contact with one or more particles comprising hydrolysable silicon and one or more lipids.

11. A method according to clause 10, wherein bringing the mRNA into contact with the one or more particles comprising hydrolysable silicon and the one or more lipids, results in an mRNA vector composition according to any of clauses 1 to 9.

12. A pharmaceutical composition comprising the mRNA vector composition according to any one of clauses 1 to 9, wherein the pharmaceutical composition is a prophylactic or therapeutic vaccine composition.

13. A pharmaceutical composition according to clause 12, wherein the mRNA has a half-life in the pharmaceutical composition at 4° C. in a phosphate buffer solution at pH7.4 of at least 3 months.

14. A pharmaceutical composition according to clause 13, wherein the mRNA has a half-life in the pharmaceutical composition at 4° C. in a phosphate buffer solution at pH7.4 of at least 6 months.

15. A pharmaceutical composition according to any one of clauses 12 to 14, wherein the pharmaceutical composition further comprises an adjuvant.

16. A pharmaceutical composition according to any one of clauses 12 to 15, wherein the pharmaceutical composition is in a form suitable for intramuscular injection.

17. A pharmaceutical composition according to any one of clauses 12 to 16, for use as a medicament.

18. A method of treating or preventing a disease or disorder, comprising: administering to a subject in need thereof a pharmaceutical composition according to any one of clauses 12 to 17.

19. A method of providing a vaccination to a subject, comprising oral (for stimulation of mucosal or gut associated lymphoid tissue MALT/GALT), intranasal, intra-dermal, ocular, subcutaneous or intramuscular administration of a pharmaceutical composition according to any one of clauses 12 to 17.

20. Use of a pharmaceutical composition according to any one of clauses 12 to 17 in the manufacture of a medicament, for example a vaccine.

### EXAMPLES

Various aspects and embodiments of the invention are now described with reference to the following non-limiting examples.

### SUMMARY

Example 1—preparation of compositions (materials and methods) and TEM imaging of them to show aggregates of Si particles therein, especially aggregates with a “branched” or “chain-like” configuration.

Example 2—stability of a chemically unmodified mRNA (Gluc mRNA, having a length of 940nt and encoding Gaussia luciferase; the open reading is understood to be formed mostly or completely of naturally occurring ribonucleosides) is improved by silicon-containing delivery vehicles, as indicated by assay binding efficiency.

Example 3—heavily modified mRNA displays poor transfection efficiency with off-the-shelf transfection reagents (Dharmafect and Lipofectamine), demonstrating the need for transfection delivery vehicles capable of stabilizing unmodified mRNA.

Example 4—silicon-containing delivery vehicles improve transfection outcomes (transfection efficiency; tolerability/non-toxicity) for AD293 cells with a chemically unmodified linear mRNA, having a 5′ Cap 1 structure and 3′ poly(A) tail (DasherGFP®, obtainable from Aldevron; the open reading is understood to be formed mostly or completely of naturally occurring ribonucleosides), compared to naked mRNA transfected using Lipofectamine 2000, and compared to identical delivery vehicles except without the silicon, after 24 h and after 48 h. The effect of storage temperature was also investigated, and it was found silicon-containing delivery vehicles' effect could be sustained even upon storage complexed with the mRNA at 37° C.

Example 5—silicon-containing delivery vehicles improve stability of DasherGFP® in bovine serum, mimicking in vivo conditions wherein mRNA is liable to degradation.

Example 6—silicon-containing delivery vehicles improve stability of Gluc mRNA in bovine serum at 37° C., mimicking in vivo conditions wherein mRNA is liable to degradation.

### Example 1: Materials and Methods: Preparation of Silicon Nanoparticles (SiNP) and SiNP-Containing Formulations

### Preparation of Silicon Nanoparticles

Single side polished silicon wafers were purchased from Si-Mat, Germany.

### Specifications of Si Wafer:

single side polished Wafer, CZ
Diameter: 150±0.2 mm
Orientation: (100)±1°
Type: p/boron
resistivity: 0.014±25% Ohmcm. Close to 5×10\hat{ }18 Atoms/cm\hat{ }3.
Primary flat: 57.50±2.5 mm
Primary flat1 Location: D<100> to {110}
Thickness: 675±15 μm
packing: Ultrapak Shipping Cassette
TTV: <=18 μm
TIR: <=5 μm

The wafers are around 40% porosity and up to 50 μm thick.

Etched silicon wafers were prepared by anodically etching Si in a 1:1 (v/v) mixture of pure ethanol and 10% aqueous HF acid for 2-10 min at an anodic current density of 80 mA/cm². After etching, the samples were rinsed with pure ethanol and dried under a stream of dry high-purity nitrogen prior to use. (All etching and cleaning reagents were clean room grade.) The silicon wafers were then manually milled using a ball mill or pestle and mortar.

The appearance before and after milling is shown in FIG. 1.

The fine powder was sieved using a Retsch™ sieve shaker AS 200 with gauge 38 μm. Uniform particle size was achieved by selection of an appropriate sieve aperture size. The particle sizes were measured by a quantachrome system and PCS (suitably obtainable from Malvern instruments). Samples were kept in a closed container until further use.

Nano silicon powder is also obtainable from Sigma Aldrich and Hefei Kaier, China. Particle size may be measured by PCS.

The size of the particles may be recorded, for example, by SEM, as shown in FIG. 2. In FIG. 2, the average nanoparticle size appears to be about 5-30 nm, with just one or two larger particles.

### Preparation of Silicon-Containing Delivery Vehicles

500 mg porous silicon nanoparticles (average diameter ca. 5-30 nm) were mixed with 250 ml ethanol and stirred using a magnet bar for 30 minutes. The solution was then centrifuged for 30 minutes at 3000 rpm. The supernatant was discarded and the nanoparticles were washed in 5 ml of distilled water and transferred to a round bottomed flask. The contents of the flask were frozen (2 hours at −25° C.). The frozen nanoparticles were freeze-dried using a freeze dryer overnight. The resultant dry powder is “activated” silicon nanoparticles. This means that any undesirable thin layer of surface oxidation, which may be present on the silicon particles, has been removed.

Meanwhile, lipid films were prepared from a solution in methanol of the relevant lipids (see Examples 2 to 6 for the types and amounts of lipids used in each respective Example), by mixing all lipids in a glass round-bottomed flask, then evaporating all solvent, with rotary evaporator in water bath, at 40° C.

Glycine was provided, as a solid powder (from Sigma Aldrich).

The silicon nanoparticles and glycine were added to the lipid film (again, see Examples 2 to 6 for amounts) together with nuclease-free water. The mixture was agitated in a water bath at 60° C. for 10 minutes, thus rehydrating the lipid film.

The resulting suspension was extruded through a polycarbonate membrane filter having 0.4 μm and 0.1 μm pore sizes. It was passed 10 times at 60° C. through each pore size.

For the preparation of complexes with mRNA, the required volumes of the “empty” silicon-containing delivery vehicle (i.e., without the mRNA) were mixed with the required volumes of mRNA stock solution (again, see Examples 2 to 6 for amounts) and the final concentration of mRNA was adjusted using nuclease-free water. The resultant samples were mixed thoroughly by gently pipetting and were incubated at room temperature for 60 min to allow for complexation to complete.

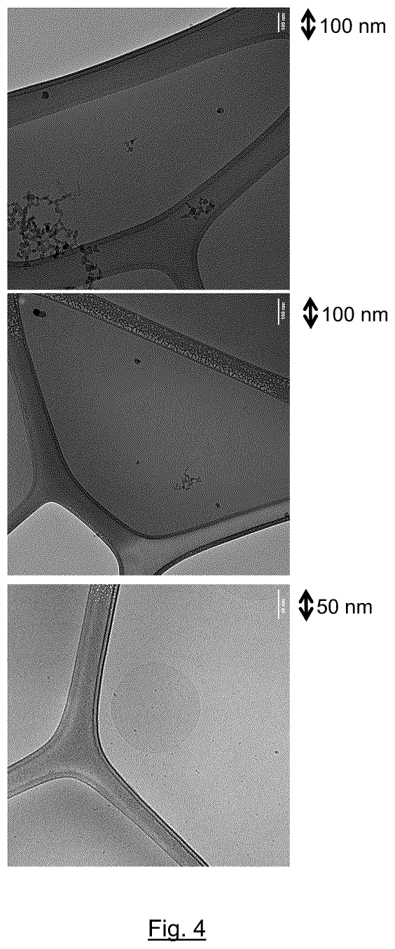

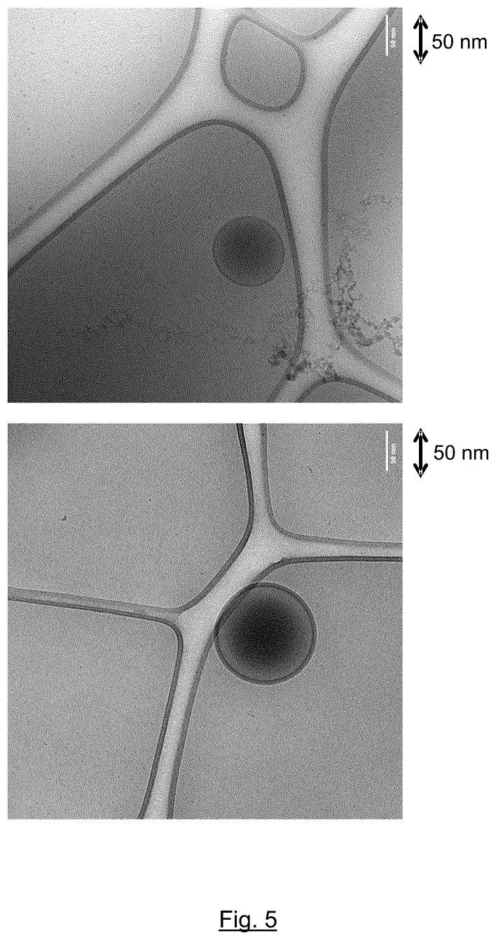

FIGS. 3, 4 and 5 are TEM images of the silicon particles with lipids and other components (FIG. 3, without nucleic acid; FIG. 4, loaded with nucleic acid; FIG. 5, as for FIG. 4 but with more heavily boron-doped silicon, compared to FIG. 4).

### Example 2: Demonstration of Benefits of Silicon on Nucleic Acid Binding Efficiency and on the Complex's Stability

A series of silicon-containing delivery vehicle formulations (produced as described in the protocol of Example 1; and with components in the amounts shown in Table 1 below) were screened to assess whether the presence of silicon exerts a beneficial effect on nucleic acid binding efficiency for non-chemically modified mRNA (and thus exerts a beneficial effect on its stability), in comparison with lipid nanoparticle (liposomal) compositions formulated with the same components, and in the same manner, as the respective silicon-containing delivery vehicle composition, but without the silicon-containing particles.

In particular, a study was performed using silicon-containing delivery vehicle formulations DSC613G, DS61G, DS6G and D6G, with a non-chemically modified mRNA, namely Gluc mRNA, having a length of 940nt and encoding Gaussia luciferase, to assess the impact of the described silicon particle-containing delivery platforms, on nucleic acid stability.

TABLE 1
Concentration of components in samples (μg/mL): the silicon-containing delivery vehicle compositions used in
Example 2 (and Examples 3 to 6); and respective liposome formulations, loaded with mRNA (or hsDNA in a repeat study)

| | Unloaded | DSC613G | | DS61G | | DS64G | | D6G | |
|---|---|---|---|---|---|---|---|---|---|
| | mRNA | Loaded | Loaded | Loaded | Loaded | Loaded | Loaded | Loaded | Loaded |
| | (control) | with Si | without Si | with Si | without Si | with Si | without Si | with Si | without Si |
| mRNA | 50 | 50 | 50 | 50 | 50 | 50 | 50 | 50 | 50 |
| SiNP | — | 100 | — | 100 | — | 100 | — | 100 | — |
| (avg. dia. | | | | | | | | | |
| 5-30 nm) | | | | | | | | | |
| DOPE | — | 240 | 240 | 240 | 240 | 240 | 240 | 240 | 240 |
| SA | — | 40 | 40 | 40 | 40 | 160 | 160 | — | — |
| DC-Chol | — | 120 | 120 | — | — | — | — | — | — |
| Gly | — | 50 | 50 | 50 | 50 | 50 | 50 | 50 | 50 |

| | | | |
|---|---|---|---|
| standard lipid base | without DC-Chol | without DC-Chol | without |
| | (amount of DOPE and SA | (amount of SA increased | DC-Chol and SA |
| | unchanged) | to compensate) | (DOPE only) |

*avg. dia. = average diameter; SiNP = silicon nanoparticles; Gly = glycine.

To estimate the binding efficiency for mRNA, the samples were centrifuged to separate unbound mRNA. The mRNA content in the supernatant fluid was measured by spectrophotometry, and the binding (entrapment) efficiency (EE) was calculated using the following equation:

$$EE \% = \frac{{OD}_{unloaded} - {OD}_{loaded}}{{OD}_{unloaded}} \times 100 \%$$

wherein OD_{unloaded} is the absorbance for the unloaded mRNA. OD_{loaded} is the absorbance for each sample L0.5 to L8.

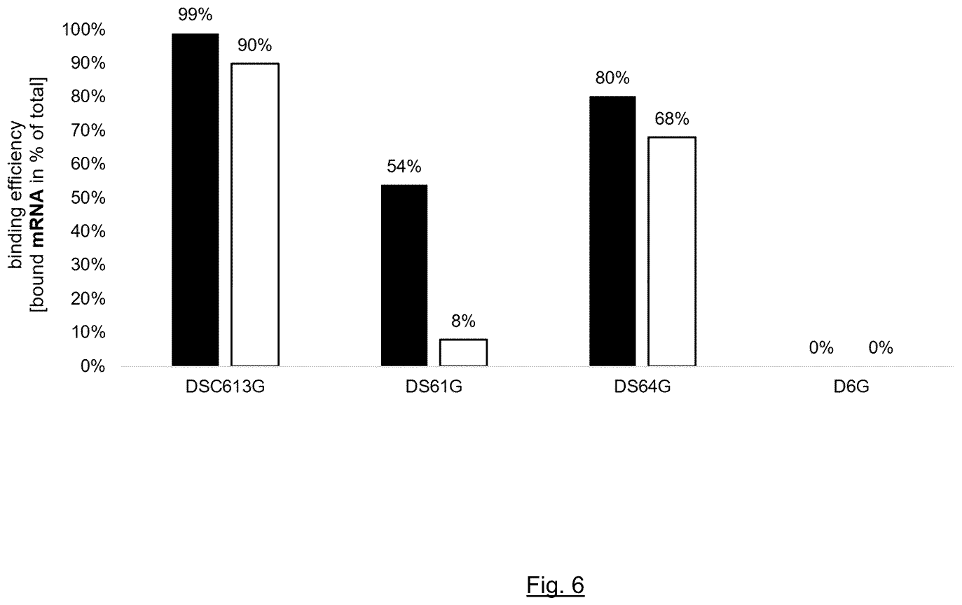

The results are shown in FIG. 6. Results for the silicon-containing compositions are shown as solid bars; whereas the liposomal compositions without silicon have the results shown as open bars. The 0% results for D6G is thought to be due to failure in the experimental procedure.

The binding efficiency (and thus, it is inferred, the stability) of the unmodified mRNA is improved when silicon particles are present.

Thus, FIG. 6 demonstrates that unmodified mRNA binding and stability are superior for formulations containing silicon particles as opposed to corresponding formulations made without the silicon particles.

### Example 3: Study of Transfection Efficacy for Chemically Modified mRNAs Encoding the Firefly Luciferase (Fluc), Namely mRNA1 and mRNA2

Two different firefly luciferase reporter mRNAs were used in this study, as shown in the table below. Both mRNAs are chemically modified, and the experiment demonstrates the potential negative impact of chemical modification on transfection and expression efficiency, thus evidencing the need for compositions capable of stabilizing unmodified mRNA.

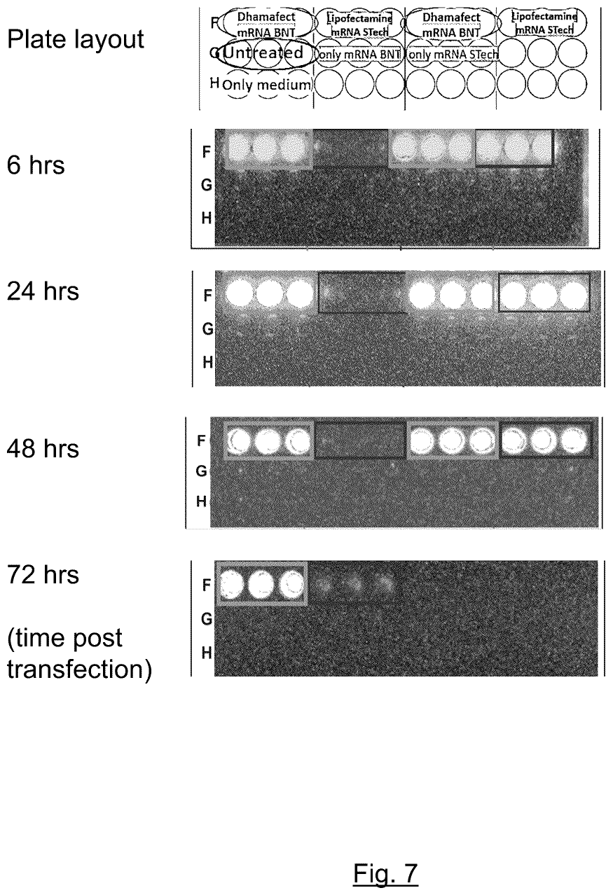

The mRNA2 (“Stratech”, or “STech”, see FIG. 7) product is modified by base methylation. The mRNA1 (also labelled “BNT”, see FIG. 7) is understood to be very heavily chemically modified; mRNA1 is more chemically modified than mRNA2.

| Reporter gene | Fluc mRNA1 (BNT) | EZ Cap Fluc mRNA2 (STech) |
|---|---|---|
| Reporter gene type | Firefly luciferase | Firefly luciferase |
| mRNA length | 2315 nucleotides | 1921 nucleotides |
| Structure | labelled as mod-Luc RNA | mRNA with 5′ Cap 1 structure and |
| | Lot no. 1836-006 | 3′ poly(A) tail, modified by 5- |
| | | moUTP |

In vitro cell transfection assays were performed with each mRNA using off the shelf transfection reagents Dharmafect and Lipofectamine.

Transfection was assessed by luciferase activity after 6, 24, 48 and 72 h post transfection.

Luciferase detection performed using:
Bright-Glo reagent kit from Promega
Hamamatsu 1394 ORCA II Deep Cooling BTA 1024 imaging system

### Assay Details

The human embryonic kidney (HEK293) cell line was used to evaluate transfection.

HEK293 cells were cultivated in Dulbecco's Modified Eagle's Medium (Euroclone, Italy) supplemented with 10% inactivated fetal bovine serum (FBS, Gibco, UK), 1% L-Glutamine (Euroclone, Italy) and 1% Pen/Strep (Euroclone, Italy), incubated under 5% CO₂ at 37° C. and passaged following standard laboratory procedures.

Twenty-four hours prior to transfection, the HEK293 cells were seeded at 1×10⁴ cells per well on 96-well black plates with transparent bottom (or 2×10⁴ cells per well for assay at the 72 h time point) in 100 μl of complete medium (without antibiotic). Cells were incubated at standard culture conditions (37° C., 5% CO₂) for 24 h.

The following day, the medium was replaced with 100 μl of transfection medium containing: 10 μl of transfection complex with 1 μg mRNA1 prepared in nuclease free water, 10 μl of serum free DMEM with 1% L-glutamine and diluted with 80 μL of complete growth medium (DMEM with 10% FBS and 1% L-glutamine). This time point was taken as the transfection time point.

Lipofectamine and Dharmafect were used, for transfection, at 10 μg/ml final mRNA1 concentration (prepared in accordance with manufacturers' protocols in serum free DMEM with 1% glutamine, and further diluted with complete growth medium prior to treatment of cells). 10 μg/ml naked mRNA1 diluted in complete medium, and no mRNA (medium only), were used as negative controls. In parallel, the EZ Cap™ Firefly luciferase mRNA (Stratech, UK) was used.

After transfection, the cells were transferred to the incubator and grown at standard conditions (37° C. with 5% CO₂) for specified times. The translation product of LUC-mRNA was detected by luciferase assay (Bright-Glo Luciferase kit, Promega, USA) performed at 6 h, 24 h, 48 h or 72 h post-treatment following the manufacturer's protocol. Briefly, the plates were equilibrated at room temperature prior to addition of 100 μl/well assay reagent and were incubated for 15 minutes at room temperature. Luciferase activity was assessed through a luminometer apparatus (Hamamatsu Photonics bioluminescence imager, Hamamatsu Photonics Italy S.R.L, Arese, Italy—integrated with Digital Camera and camera controller, C4742-98—Software Wasabi, v. 1.5, Hamamatsu Photonics).

### Results

FIG. 7 shows transfection efficiency at each time point. A bright dot shows effective transfection and the intensity of the dot corresponds to the extent of transfection.

### 6 h Post Transfection

At 6 h post-transfection, Dharmafect was able to transfect cells both mRNA models whilst Lipofectamine was effective only with the Stratech mRNA.

### 24 h Post Transfection

At 24 h post transfection, Dharmafect was able to transfect both mRNA models whilst Lipofectamine was effective only with the Stratech mRNA at the same assay conditions.

### 48 h Post Transfection

At 48 h Dharmafect was able to transfect both mRNA models whilst Lipofectamine was effective only with the Stratech mRNA at the same assay conditions.

### 72 h Post Transfection

Dharmafect showed a signal only when complexed with mRNA1, while with mRNA2 the signal was lost for both Dharmafect and Lipofectamine.

### CONCLUSION

Despite Dharmafect and Lipofectamine being generally considered to be effective transfection agents, the transfections assessed were only partially successful due to the heavily modified nature of the mRNAs, especially mRNA1.

### Example 4. Demonstration of Superior mRNA Transfection when Unmodified mRNA is Formulated with Silicon

### Study Aim

This study used a commercially available linear mRNA product—DasherGFP® (see https://www.aldevron.com/products/mrna-products/dasher-gfp-mrna).

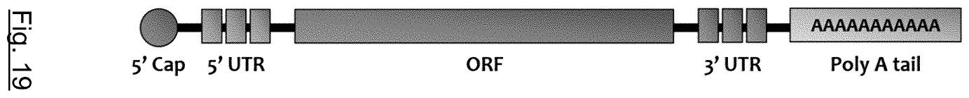

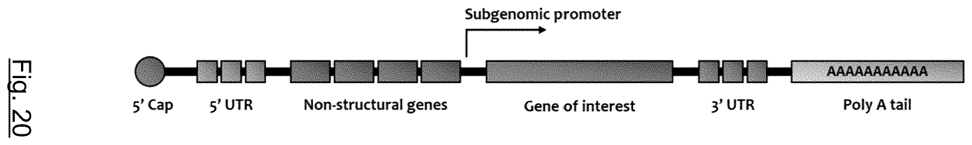

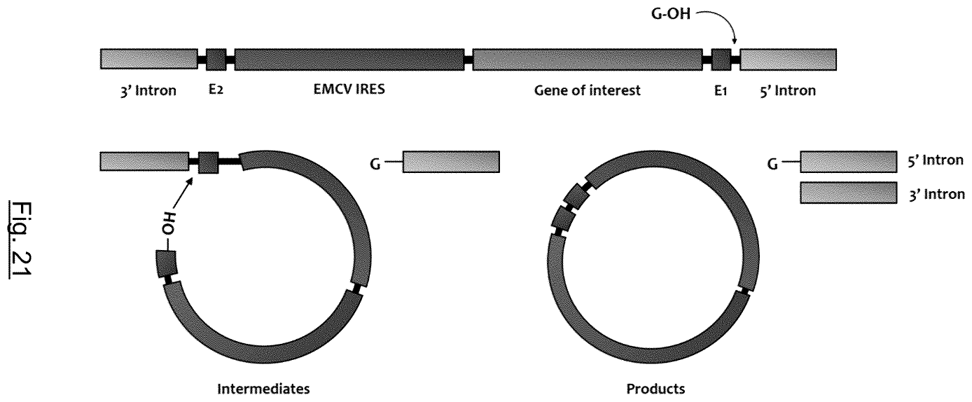

Based on the supplier's information, the DasherGFP® sequence is optimised for expression in mammalian cells, hence the inclusion of a 5′ Cap 1 structure and 3′ poly(A) tail, mimicking the natural structure of mature linear eukaryotic mRNA, as shown in FIG. 19. It contains no chemical modifications. Thus, DasherGFP® provides a good (and generalisable) model for chemically unmodified mRNA in accordance with the present disclosure.

This study included an investigation of the effect of storage temperature on the transfection efficiency of silicon-containing delivery vehicle compositions. A reporter gene expression assay was used to evaluate the transfection of DasherGFP® mRNA-loaded silicon-containing delivery vehicle formulations (“Bio-Courier GEN-AVE-1C, batch MIV0015”) (i) in solution (sshLNP); and (ii) when embedded in a sodium hyaluronate hydrogel (ColHySilic), following storage at (a) room temperature, (b) 37° C. or (c) 4° C., to study the effect of temperature on the complex stability and cellular behaviour of carrier system. DasherGFP® mRNA (Aldevron) encodes a fluorescent protein and is optimized for expression in mammalian cells for studying transfection and expression. The expressed protein yields bright green fluorescence allowing for direct detection and analysis of mRNA delivery and expression.

The Bio-Courier GEN-AVE-1C (batch MIV0015) composition is prepared in accordance with the protocol of Example 1 and has the following components:
7.75 mg DOTAP
8.25 mg DOPE
1 mg Si nanoparticles
0.5 mg glycine
1 mg trehalose

For comparison, GEN-AVE-WS was prepared, having the same components as Bio-Courier GEN-AVE-1C (batch MIV0015), but without silicon nanoparticles.

Also used, as a further control, was naked mRNA stored at (a) room temperature, (b) 37° C. or (c) 4° C.;

following which transfection to cells was carried out by Lipofectamine 2000, a conventional transfection reagent.

### Methods

Bio-Courier GEN-AVE-1C (batch MVI0015, produced by tangential flow filtration process) was combined with DasherGFP® mRNA at 1:12 weight ratio (mRNA to total lipid mass).

For comparison, the same complexation was performed for the described liposome-only formulation, equivalent to GEN-AVE-1C without silicon nanoparticles (GEN-AVE-1C-WS) to investigate the role of Silicon matrix.

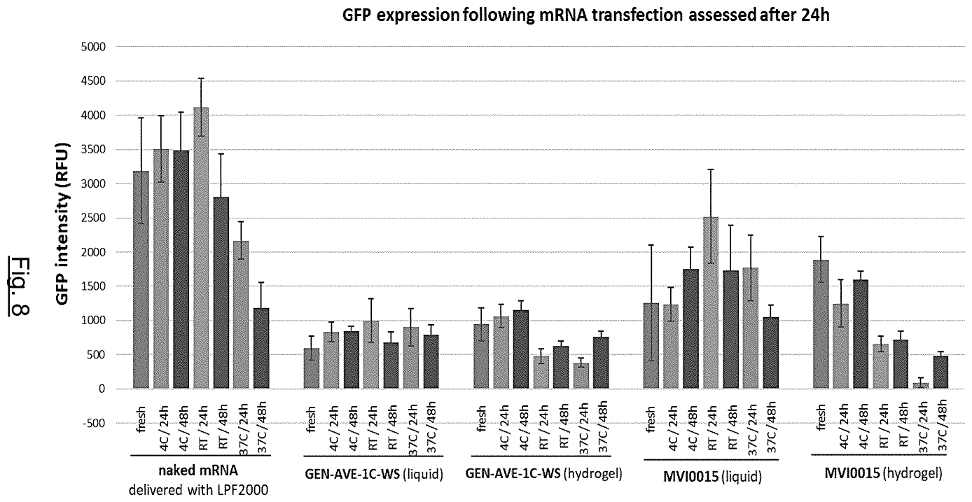

In the Figures (see, e.g., FIG. 8), Bio-Courier GEN-AVE-1C (batch MVI0015) is labelled as “MVI0015”, while GEN-AVE-1C-WS is labelled as “GEN-AVE-1C-WS”.

Upon complexation (i.e., after mRNA complexation) one set of formulations was mixed with 2% sodium hyaluronate hydrogel in 1:1 ratio, whereas the other set was diluted 1:1 with nuclease free water and used as liquid samples.

The final preparations were aliquoted and stored either at standard refrigerated conditions (4° C.), at room temperature or at 37° C.

As a control, naked mRNA was prepared in nuclease-free water, and stored at the same temperatures.

For transfection, the naked mRNA was combined with conventional Lipofectamine 2000 (following manufacturer's protocol), as described above.

Prior to transfection, FAD293 cells were seeded on a 96-well black plate with optical clear bottom at 5,000 cells/well seeding density and grown in a cell incubator at 37° C. with 5% CO₂ overnight.

The cells were then transfected with Bio-Courier GEN-AVE-1C (batch MVI0015), GEN-AVE-1C-WS, or Lipofectamine 2000.

In brief, for transfection to cells, the various DasherGFP® complexes were diluted in cell culture medium and added to the cells in n=6 replicates at final 1 μg/mL mRNA concentration (100 ng per 100 μL growth medium per well). Untreated cells were grown in a complete growth medium as a control.

At designated time points of 24 h and 48 h post-transfection, GFP fluorescence and Hoechst staining of the cell cultures were observed.

In brief, after 24 and 48 h of incubation at standard culture conditions, the cells were washed with PBS and stained with NucBlue Live reagent containing Hoechst 33342 fluorescent nuclear dye (ThermoFisher Scientific, cat. R37605). Hoechst fluorescence intensity was measured at 355/460 nm Ex/Em using well-scanning mode on a plate reader. For assessing the level of GFP expression following DasherGFP® mRNA transfection, GFP fluorescence intensity was measured at 485/520 nm Ex/Em using BMG Labtech FLUOstar OPTIMA plate reader and cells were imaged using Zeiss Axio Scope A1 fluorescence microscope equipped with green (GFP) and blue (Hoechst) filter.

### Results

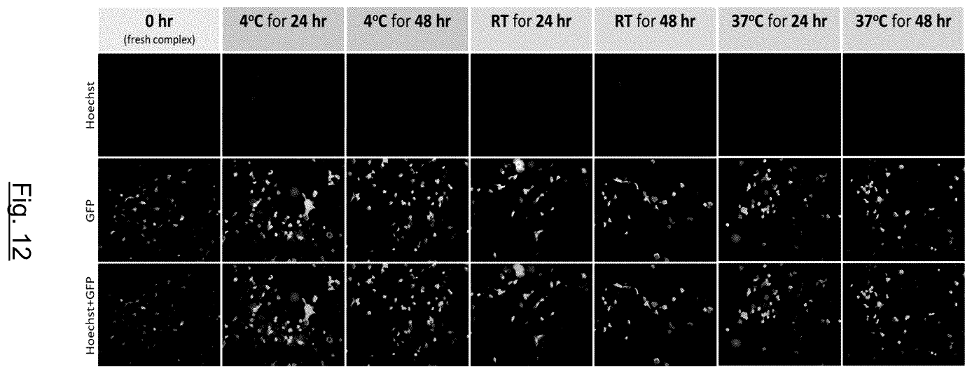

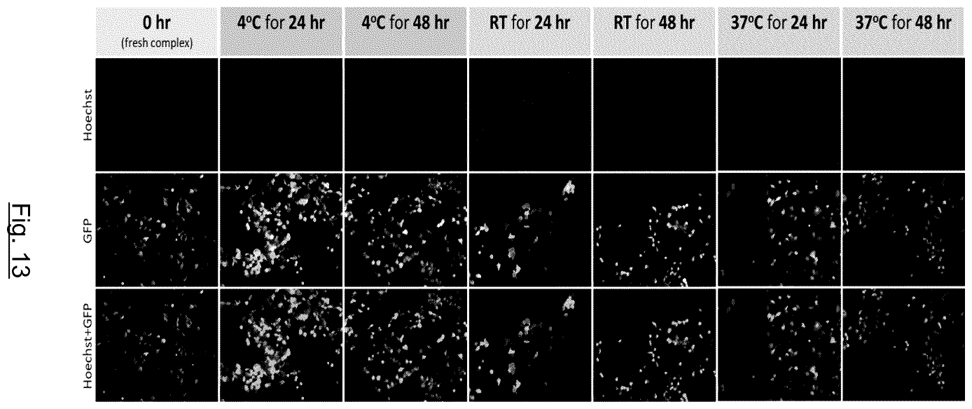

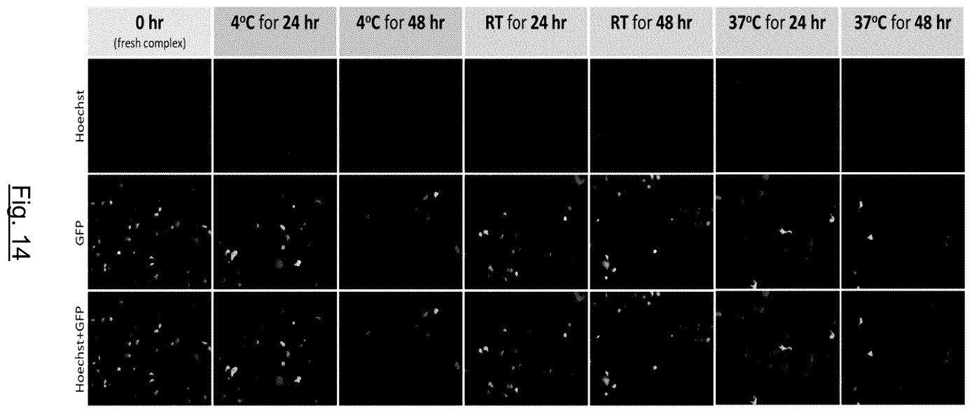

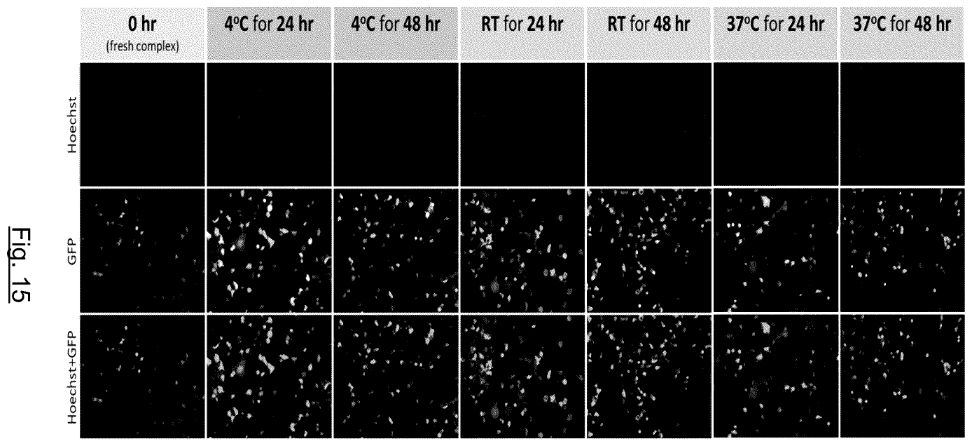

The results are shown in FIGS. 8 to 15.

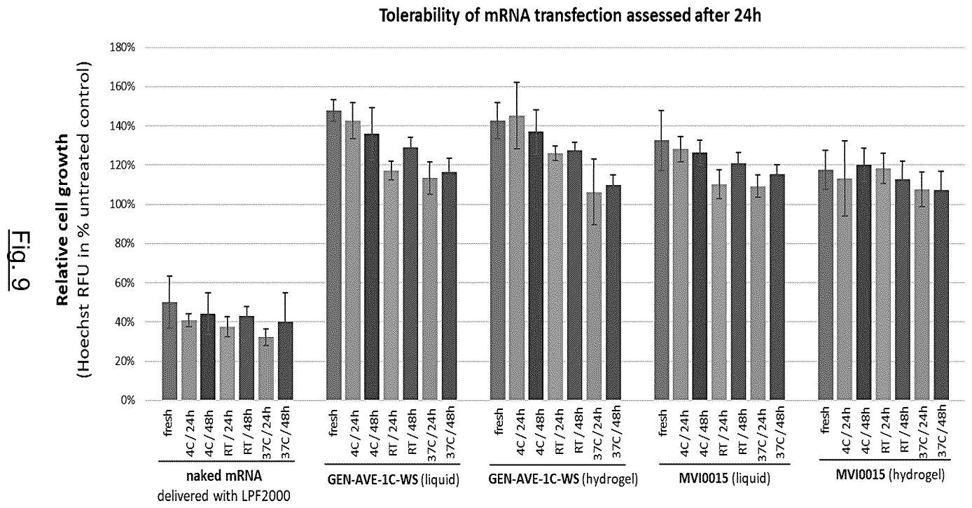

FIG. 8 shows that at 24 hours after transfection, naked mRNA (transfected using Lipofectamine 2000) had the strongest GFP signal, but only for mRNA stored at low or room temperature. mRNA stored at high temperature showed a much weaker signal, it is thought due to mRNA degradation. Moreover, FIG. 9 shows strikingly that the Hoechst fluorescence was reduced for the naked mRNA (Lipofectamine 2000) samples, indicating poor tolerability. This may be due to the toxicity of conventional liposomal delivery vehicles, such as Lipofectamine.

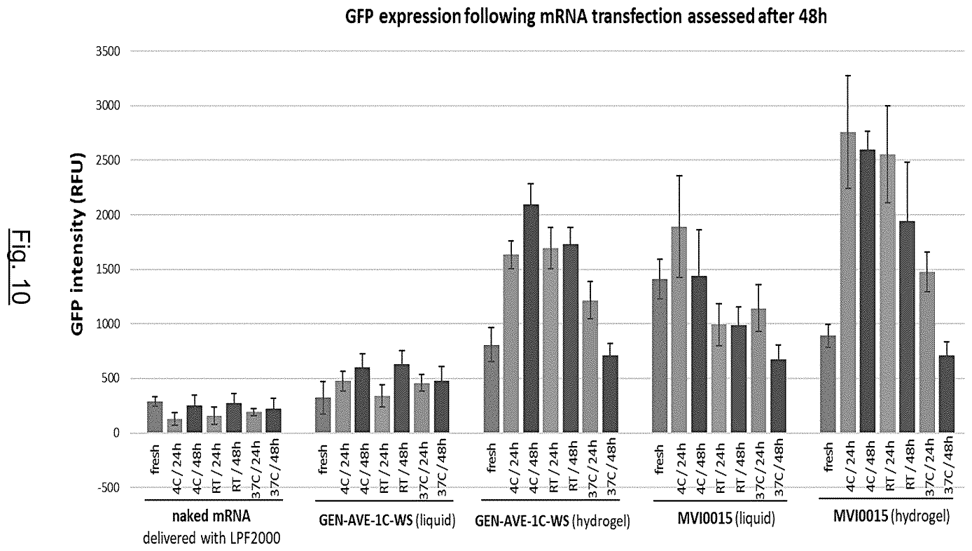

As shown in FIG. 10, by 48 h after transfection, Bio-Courier GEN-AVE-1C (batch MVI0015) (the silicon-based delivery system) appeared to be more efficient in mRNA delivery than the equivalent liposomal formulation without silicon (GEN-AVE-1C-WS), in a like-for-like comparison (GEN-AVE-1C in solution vs. GEN-AVE-1C-WS in solution; and GEN-AVE-1C in hydrogel vs. GEN-AVE-1C-WS in hydrogel).

Moreover, Bio-Courier GEN-AVE-1C (batch MVI0015) was better able to maintain transfection efficiency regardless storage conditions and including following storage at 37° C., than equivalent liposomal compositions in a like-for-like comparison (GEN-AVE-1C in solution vs. GEN-AVE-1C-WS in solution; and GEN-AVE-1C in hydrogel vs. GEN-AVE-1C-WS in hydrogel). Additionally, naked mRNA delivered using Lipofectamine 2000 was especially affected by exposure to 37° C. (decreased GFP expression observed after storage).

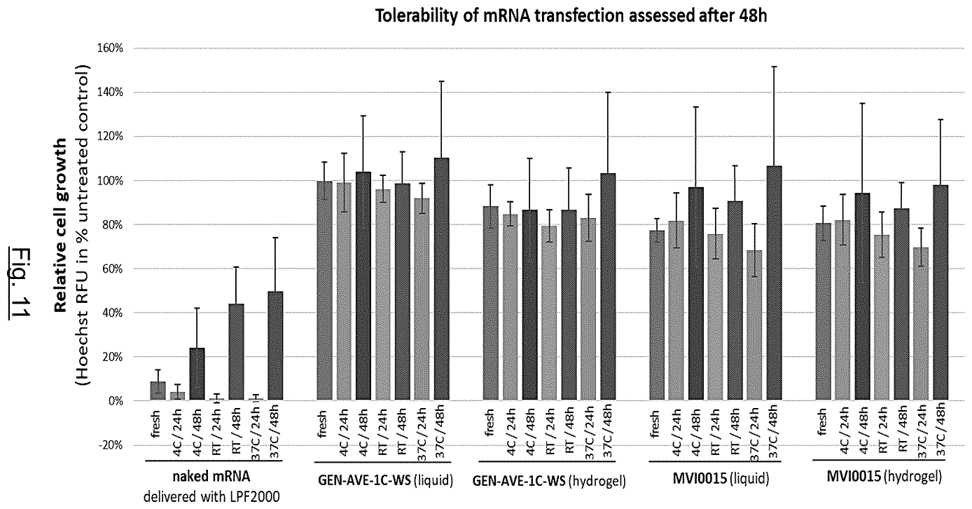

FIG. 11 shows that at 48 h after transfection, tolerability of transfection with Bio-Courier was sustained. Whereas, under the same conditions, mRNA delivered using Lipofectamine showed high cell toxicity: 50-100% cell death was observed for the cells wherein mRNA was delivered using Lipofectamine, compared to untreated control cells. The GFP signal for mRNA delivered using Lipofectamine was also very low at 48h.

The steady increase in GFP expression from 24 h to at 48 h post transfection with Bio-Courier GEN-AVE-1C (batch MVI0015) suggests sustained protein expression and stability under in vitro conditions.

Interestingly, embedding of formulations in sodium hyaluronate appears to have improved their resistance to thermal stress: an increased GFP expression was observed for all hydrogel samples, when compared with those in solution.

### Summary of Findings

This cell transfection study suggested good stability of Bio-Courier/mRNA complex upon storage at refrigerated, ambient and higher temperature. Freshly complexed and incubated Bio-Courier formulation showed a moderate mRNA transfection efficiency and was well tolerated by the cells.

Embedding of the formulation in sodium hyaluronate improved its resistance to thermal stress and an increased protein expression was observed for hydrogel samples when compared with liquid version, especially in case of liposomal GEN-AVE-1C-WS formulated without SiNP that supported the role of silicon in stabilisation of sshLNP.

### Example 5. Demonstration of Stabilisation of Unmodified mRNA in Serum by Si-Containing Delivery Vehicles

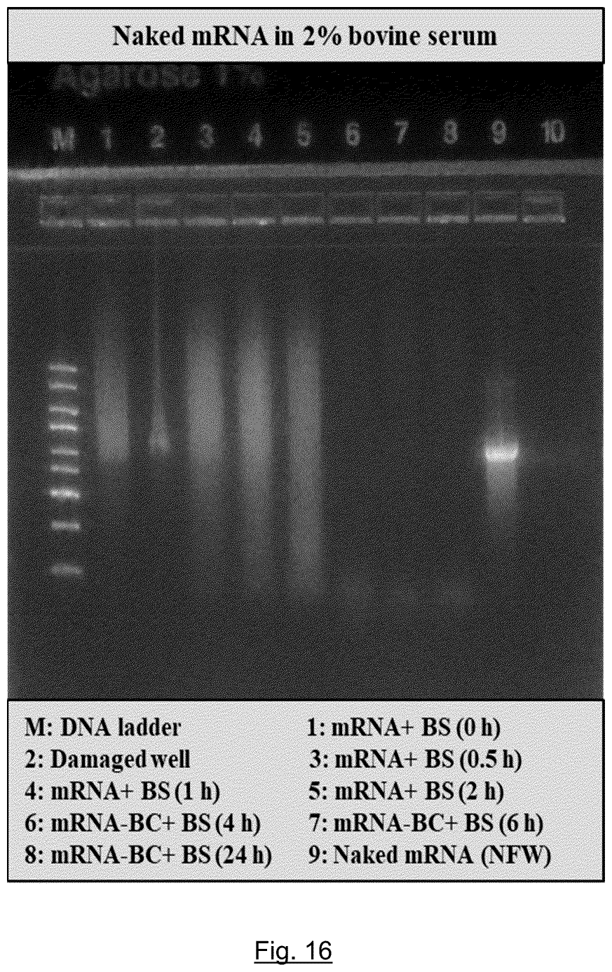

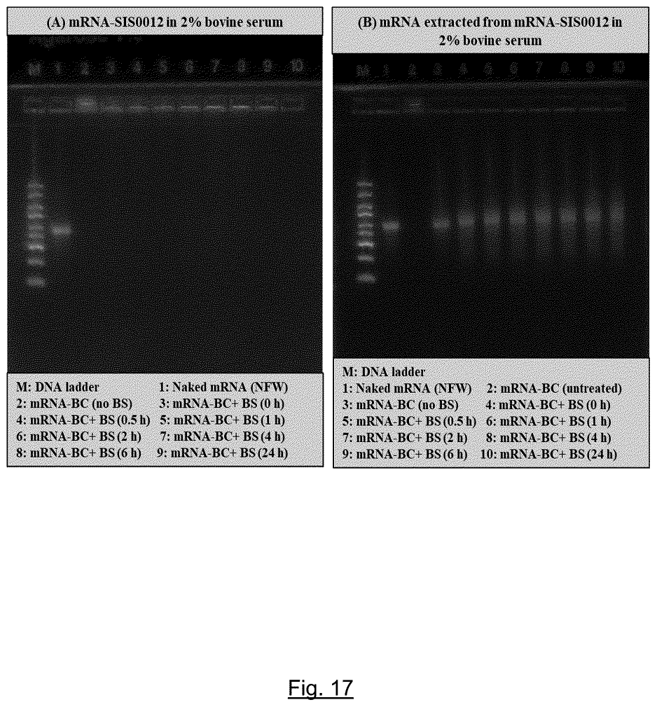

FIGS. 16 and 17 show the results of an experiment to determine stabilisation, by means of silicon-containing delivery vehicles in accordance with all aspects of the invention, in 2% bovine serum, closely mimicking in vivo conditions.

As the silicon-containing delivery vehicle, the following “SIS0012” Bio-Courier formulation, prepared in accordance with the protocol of Example 1, was used:
7.25 mg DOTAP (1.45 mL)
7.3 mg DOPE (1.46 mL)
1.45 mg DSPE-PEG2000 (0.29 mL)
1 mg Si (undoped)
1 mg trehalose
0.5 mg glycine
nuclease-free water in an amount sufficient to make the formulation up to 10 mL.

### Method

Aliquots of mRNA (DasherGFP® mRNA from Aldevron), as described in Example 4) either naked or loaded onto Si-containing SIS0012, were incubated in the presence of 2 or 10% fetal bovine serum (FBS) in nuclease free water at room temperature over time. Samples were analysed at intervals by electrophoresis on a 1% E-Gel agarose gel using a Power Snap Electrophoresis system in accordance with the manufacturer's standard protocol.

### Results

As can be seen in FIG. 16, when using a control sample of naked chemically unmodified DasherGFP® mRNA, incubated in the 2% bovine serum, the mRNA is degraded. This is evidenced by its smearing on the gel.

While FIG. 16 shows smearing for the mRNA upon first contact with serum (i.e, at 0 h), for this sample no degradation has yet occurred, as the travelled distance is similar to that of the untreated mRNA control (in nuclease-free water). However, fragmentation of mRNA starts appearing from as early as 0.5 h after incubation with serum, as evidenced by the longer distance travelled by fragments of mRNA which have now formed. Fragmentation increases over time. From 4 h onwards, complete degradation of mRNA occurs, indicated by the disappearing of the smeared band and the presence of a very weak signal towards the bottom of the gel, which is lower than the smallest fragment in the DNA ladder. These data clearly indicate susceptibility of the unmodified mRNA to nuclease enzymes present in bovine serum.

In sharp contrast, FIG. 17 shows stabilisation of the mRNA, in serum, by the SIS0012 delivery vehicle.

Compared to FIG. 16, FIG. 17(B) shows that mRNA extracted from the composition is mostly not degraded, and a band remains even at 24 hours after incubation in serum. Meanwhile, FIG. 17(A) confirms tight binding of the mRNA by the SIS0012 composition, as evidenced by the lack of bands in wells 3 to 10.

### Example 6. Further Demonstration of Stabilisation of Unmodified mRNA in Serum by Si-Containing Delivery Vehicles, Even at Higher Temperatures

### Study Aim

To determine if mRNA loaded onto silicon-containing delivery vehicle DSC613G (see Table 1 of Example 2 for a description of this delivery vehicle; it was prepared in accordance with the protocol of Example 1) would be protected from nuclease degradation, uncomplexed (naked) mRNA and DSC613G-complexed mRNA samples were analysed by gel electrophoresis following exposure to serum containing RNA nucleases, mimicking in vivo conditions; similarly to Example 5, but with a different unmodified mRNA and at an elevated temperature of 37° C.

### Method

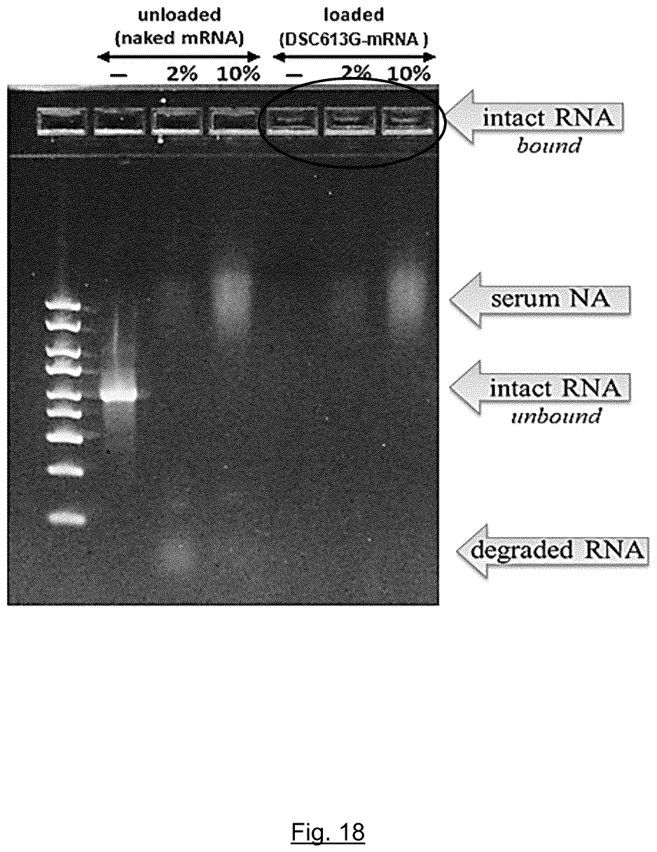

Aliquots of mRNA (as in Example 2, Gluc mRNA having length of 940nt and encoding Gaussia luciferase was used as the unmodified mRNA) either naked or loaded onto Si-containing DSC613G were incubated in the presence of 0% (control), 2% or 10% foetal bovine serum (FBS) in nuclease free water for 1 h at 37° C. (see labels of FIG. 18:—for 0%, followed by 2% and 10%). After the incubation period, the samples were placed on ice to inhibit further nuclease degradation and analysed immediately by electrophoresis on a 1% E-Gel agarose gel using a Power Snap Electrophoresis system in accordance with the manufacturer's standard protocol. The gel was visualized after 8 minutes with a built-in camera device.

### Results

As shown in FIG. 18, naked mRNA was almost entirely degraded within 1 hr when incubated at 37° C. with buffer containing 2% FBS or 10% FBS (only a weak smearing was detected at shorter fragment size after incubation), compared to untreated (0% FBS) mRNA control.

In contrast, no degraded fragments were detected for mRNA complexed with DSC613G, as evidenced by the blank next to the label “degraded NA” for the DSC613G-mRNA samples at 0, 2 or even 10% FBS. Instead, the majority of the nucleic acid remained intact within the particle complex following nuclease treatment, meaning it was trapped in the loading wells visible in top of the gel image, shown in the white-circled area of FIG. 18. This suggests that most mRNA bound to/in DSC613G was protected from nuclease degradation.

Note that, as labelled, FIG. 18 also shows a weak band towards the middle of the plate for mRNA of the serum itself, for both naked mRNA and mRNA complexed with DSC613G.

### Summary of Findings

Silicon-containing delivery vehicles, prepared in accordance with the present invention and formulated with chemically unmodified mRNA, promoted resistance of the mRNA to nuclease degradation, even in serum at 37° C. Moreover, the silicon-containing delivery vehicles appear to be non-toxic, comparing favourably to Lipofectamine 2000. Stable transfection of cells was also evidenced when mRNA was delivered to them by the silicon-containing delivery vehicles, confirming not only stabilisation but also transfection efficiency.

Without wishing to be bound by theory, it is thought that complexation of chemically unmodified mRNA with the silicon-containing delivery vehicles may provide protection from degradation, especially nuclease-mediated degradation. This may be due to steric effects and/or due to charge-charge interactions.

In particular, it is thought that stabilisation of chemically unmodified mRNA may be assisted by aggregation of the particles comprising hydrolysable silicon, as shown in the transmission electron microscope (TEM) images of FIGS. 3 to 5. When the one or more aggregates are or comprise chains of the particles, such chains may extend into the interior of lipid structures. In this way, the chains May provide a route for the mRNA to be better encapsulated into lipid. Without wishing to be bound by theory, it is thought that this may shield the mRNA from degradation, especially by shielding it from enzymes (especially, in vivo) and preventing or reducing mRNA molecules being available to react with water molecules. It is thought that as the silicon particles degrade over time, mRNA may be released, thus enabling protection of the mRNA until it reaches a target site for release; hence the transfection efficiency displayed in Examples 4 to 6.

1. An mRNA vector composition comprising:
mRNA;
one or more particles comprising hydrolysable silicon; and
one or more lipids,
characterised in that the mRNA is mRNA which is not chemically modified.

2. An mRNA vector composition according to claim 1, wherein at least about 75% of ribonucleosides in the mRNA have the same molecular structure as naturally occurring ribonucleosides.

3. An mRNA vector according to claim 1, wherein the mRNA has no non-naturally occurring secondary structure.

4. An mRNA vector composition according to claim 1, wherein the mRNA comprises:
a protein-encoding open reading frame;
a 5′cap structure;
a poly(A) tail; and
one or more untranslated regions.

5. (canceled)

6. An mRNA vector composition according to claim 4, wherein the open reading frame is formed of ribonucleosides each having the same or substantially the same molecular structure as a ribonucleoside of an open reading frame of a naturally occurring mRNA, except for the modification of at least one uridine if uridine is present.

7. An mRNA vector composition according to claim 1, wherein, the mRNA vector composition comprises one or more aggregates of the particles comprising hydrolysable silicon.

8. An mRNA vector composition according to claim 7, wherein within the one or more aggregates the particles comprising hydrolysable silicon have an average diameter in a range of from about 1 nm to about 50 nm.

9. (canceled)

10. An mRNA vector composition according to claim 7, wherein the one or more aggregates is or are embedded in one or more lipidic structures.

11. An mRNA vector composition according to claim 7, wherein mRNA is non-covalently bound to the particles in the one or more aggregates.

12. An mRNA vector composition according to claim 7, wherein the one or more lipidic structures are or comprise one or more of: liposomes, incomplete liposomes, micelles, incomplete micelles, and lipid globules.

13. An mRNA vector composition according to claim 1, wherein the mRNA, when isolated from the particle(s) and lipid(s), has a half-life in vivo of under about an hour.

14. An mRNA vector composition according to claim 4, wherein the open reading frame of the mRNA encodes an antigen of a pathogen, optionally wherein the open reading frame of the mRNA encodes:
a spike protein antigen of SARS-CoV-2;
a viral protein related to a virus that causes a disease;
a bacterial protein related to a disease causing bacterium;
a parasitic protein related to a disease causing parasite;
a fungal protein related to a disease causing fungal species;
a tumour-associated antigen or a tumour-specific antigen;
an allergen; or
a modulator of an immune disease, an autoimmune disease, or an inflammatory disease.

15. An mRNA vector composition according to claim 1, further comprising one or more amino acids; and/or one or more non-reducing disaccharides.

16. An mRNA vector composition according to claim 1, wherein the one or more lipids are or comprise (i) one or more ionisable lipids; and/or (ii) one or more PEGylated lipids.

17. A formulation comprising a hydrogel and the mRNA vector composition according to claim 1.

18. A method of increasing the stability of a mRNA molecule which is not chemically modified, comprising the step of bringing it into contact with one or more particles comprising hydrolysable silicon and one or more lipids.

19. (canceled)

20. A pharmaceutical composition comprising the mRNA vector composition according to claim 1, wherein the pharmaceutical composition is a prophylactic or therapeutic vaccine composition.

21. A pharmaceutical composition according to claim 20, wherein the mRNA has a half-life in the pharmaceutical composition at 4° C. in a phosphate buffer solution at pH7.4 of at least 3 months.

22. (canceled)

23. A pharmaceutical composition according to claim 20, wherein the pharmaceutical composition further comprises an adjuvant.

24. (canceled)

25. (canceled)

26. A method of treating or preventing a disease or disorder, comprising: administering to a subject in need thereof a pharmaceutical composition according to claim 20.

27. A method of providing a vaccination to a subject, comprising oral, intranasal, intra-dermal, ocular, subcutaneous or intramuscular administration of a pharmaceutical composition according to claim 20.

28. (canceled)

29. The mRNA vector composition according to claim 15, wherein the amino acid is glycine.

30. The mRNA vector composition according to claim 15, wherein the more non-reducing disaccharide is trehalose.
